(19) United States
(12) Patent Application Publication
(10) Pub. No.: US 2024/0004969 A1
(43) Pub. Date: Jan. 4, 2024

(54) WATERMARKING OF GENOMIC SEQUENCING DATA

(71) Applicants: Children's Hospital Los Angeles, Los Angeles, CA (US); University of Southern California, Los Angeles, CA (US)

(72) Inventors: Xiaowu Gai, La Canada-Flintridge, CA (US); Alex Ryutov, Playa Vista, CA (US); Tatyana Ryutov, Playa Vista, CA (US)

(73) Assignees: Children's Hospital Los Angeles, Los Angeles, CA (US); University of Southern California, Los Angeles, CA (US)

(21) Appl. No.: 17/918,824

(22) PCT Filed: Apr. 21, 2021
(86) PCT No.: PCT/US2021/028480

§ 371 Date: Oct. 13, 2022

Related U.S. Application Data
(60) Provisional application No. 63/011,838, filed on Apr. 17, 2020.

(51) Int. Cl. G06F 21/16; G06F 21/60

(52) U.S. Cl. CPC G06F 21/16; G06F 21/602; G06F 2221/2107

(57) ABSTRACT

Examples are described for dynamically applying a digital watermark to a file, such as a dataset of genomic sequencing data. In one example, a method of dynamically applying a watermark to at least a portion of a file includes generating a first random seed, generating an ordered pseudorandom set of integers, generating a second random seed, selecting, using the second random seed, a subset of the ordered pseudorandom set of integers, the subset corresponding to identifiers of data locations in the file, and modifying data at data locations in the file corresponding to at least a portion of the identifiers included in the subset to generate a watermarked file. The genomic data file may be an ordered Binary Alignment Map (BAM) file storing sequencing data or a Variant Call Format (VCF) file or a list of variants storing genomic variation data.

### CROSS REFERENCE TO RELATED APPLICATION

This application claims the benefit of U.S. Provisional Application No. 63/011,838, entitled “Watermarking of Genomic Sequencing Data” and filed Apr. 17, 2020, which is herein incorporated by reference in its entirety.

### BACKGROUND

The advent of next-generation sequencing (NGS) technologies led to the emergence of genomic medicine, which uses the genomic information to understand disease mechanisms and to guide patient care, such as for diagnostic, prognostic and therapeutic decision-making. As part of it, huge amount of genomic sequencing data have been generated for both research and clinical purposes with drastic more such data anticipated in the future. Genomics has been compared with other major sources of Big Data including astronomy, and may be considered the most demanding in terms of all four major aspects of Big Data, namely, data acquisition, storage, distribution, and analysis with the astronomical, or rather genomical, growth of DNA sequencing in terms of the overall sequencing capacity but also the number of human genomes sequenced each year and cumulatively.

Biomedical research has benefited tremendously from the genomical growth of sequencing capacity. For example, cancer is considered a genetic disease. Using pediatric cancer as an example, pan-cancer analyses of pediatric tumors reveal a spectrum of nuclear somatic DNA alterations that vary by tumor type, and at least 8.5% of pediatric cancer patients have germline mutations in cancer predisposition genes. The patterns of these genomic alterations are distinctly different from one tumor type to another and one patient from another, which have been shown to be of diagnostic, prognostic and therapeutic importance and implications. For example, a comprehensive next-generation sequencing panel, OncoKids, was developed for pediatric cancers, which has demonstrated significant clinical utility in two years since its launch, with clinically significantly findings found in two thirds of over 1000 patients tested. Clinical exome sequencing tests, similarly, allowed for identification of pathogenic cancer predisposition variants in 8/106 (7.5%) patients tested. Such findings have all been enabled and empowered by the advent of massively parallel sequencing technologies, which led to 1 million fold decrease of the cost of sequencing a human genome since 2003, when the human genome project was completed. These genomic technologies have led to tremendously improved understanding of cancer etiology which, however, is only possible when the researchers and the patients are willing to share the genomic data. Again using research experience as an example, the landscape of germline and somatic mitochondrial DNA mutations in pediatric cancers were able to be established from mining the matched tumor-normal whole genome sequencing data of 621 pediatric cancer patients, collected and shared by the St. Jude's Children's Hospital instead based on these patients informed consent.

With the success of the 1000 Genomes Project, the Cancer Genome Atlas program, the International Cancer Genome Consortium (ICGC), to name a few, and many large national and international population-scale genomics initiative such as the Genomics England, there has been little doubt about the benefits and the importance of sharing of genomic data. Along the way, there have been many associated challenges, however, including but not limited to technical challenges with standards, capabilities, and performance For example, along with power and excitement, the tsunami of genomic data, also presents desperate needs for advanced informatics methodologies to facilitate genomic data sharing and to address the associated challenges, both technical and also from the legal and ethical points of view.

One such challenge relates to privacy concerns regarding access to and usage of the genomic data. The genomic sequencing data is deemed Personal Health Information (PHI) according to the Health Insurance Portability and Accountability Act (HIPAA) Privacy Rule, and also the General Data Protection Regulation recently established by the European Parliament and Council of the European Union. Since the genomic sequencing data could reveal the person's risks for various diseases, such as cancers and heart diseases, privacy concerns have been raised because of potential inappropriate use of the genomic sequencing data. While the Genetic Information and Nondiscrimination Act of 2008 (GINA) has made it illegal for health insurers or employers from discriminatory use of the genomic sequencing data, there is a lack of computational methodologies for an individual a) to protect his/her privacy risks while still benefiting from genomic medicine, b) to participate in genomic research which requires sharing genomic data for the general good of the society.

On the other hand, informed consent is now the essential component of any modern biomedical research involving human subjects. The notion of informed consent emerged after decades of atrocities, followed by tremendous efforts to address the problem that resulted in The Nuremberg Code, The Declaration of Helsinki, the Common Rule, and the Belmont Report. It is now a key component of any modern biomedical research involving human subjects. Information, comprehension and voluntariness are the three pillars that define informed consent in general as the full disclosure of the nature of the research and involvement of the participant, adequate comprehension for the participant, and the participant's voluntary choice to participate or not. The conventional paper-based and static consent for a study with clearly specified goals and end points, however, could not meet the complexity and challenges of the emerging genomic research, which typically requires combining data sets from multiple studies, performing analyses that are not specified by each study at the time of consent, and making unanticipated discoveries. As such, dynamic consent is a new concept or a new consent model that is gaining support in the field. It engages all three pillars of informed consent, namely information, comprehension and voluntariness. It does so by providing a centralized computational platform that allows personalized online consent such that the consent by a potential participant can be a) done in real-time, b) granted to any researchers of choice, c) for any duration of choice, and d) for any purposes of choice. Dynamic consent promises even more granular controls of genomic data sharing by allowing the participants to control what specific portion of their genomic data can be shared, such as hiding sensitive data in specific disease genes like APOE, BRCA1/2 and other cancer predisposition genes, or neuropsychiatric disease genes.

Alternative to any consent model is the ownership-based governance. The patients or participants of any genomic study ultimately own their data, and should have the governance of the data, which includes the right to control the data and also the right to assess the value of the data, with value being economic or intellectual. This provides the ultimate and most granular control of the participant's data but requires a distributed model that 1) is participant-centric, 2) does not require any centralized management, and 3) provides the fine-grained control of the participant's data. This model comes with significant technical challenges for the participants: a) to control what (portion of) data to share, with whom and for what duration, b) to track or trace data access, c) to prevent unauthorized access, d) to prevent or deter illicit duplication and usage of the data, and e) to potentially benefit financially from sharing the data.

Either dynamic consent or ownership-based governance of accessing or sharing the genomic sequencing data, however, requires robust informatics tools to enable and to facilitate, in order to deal with the associated complexity while ensuring privacy preservation. Such algorithms or tools are severely lacking. For example, there is currently a significant lack of computational or informatics tools to enable the implementation of any real ownership-based governance. Currently, once the genomic sequencing data of an individual is shared with an entity, the individual does not have control over how the data is used and cannot verify the proper usage of the data. This makes executing dynamic consent or honoring data owner's privacy concerns extremely challenging. As an example, the General Data Protection Regulation recently established by the European Parliament and Council of the European Union, clearly defines the right of a participant of any study to revoke the consent. As it is now, however, once a participant consents to a study, which later combines and shares data with other studies, there is no practical way to erase the data and to prevent it from being used for other purposes in the future. Furthermore, there is no practical way to track the distribution of the data and identify usage of the data that does not comply with the consent provided by the participant.

### SUMMARY

This disclosure addresses the above-described challenges by providing a unified set of algorithms for watermarking genomic sequencing and variant data. Together with a dynamic privacy preserving encryption algorithm (examples of which are disclosed in U.S. Provisional Application No. 62/859,575, filed Jun. 10, 2019, and entitled “Dynamic Encryption/Decryption of Genomic Information” [hereinafter “the '575 disclosure”] and U.S. Provisional Application No. 62/891,830, filed Aug. 26, 2019, and entitled “Watermarking of Genomic Sequencing Data” [hereinafter, “the '830 disclosure”], each of which is hereby incorporated by reference in its entirety), these watermarking algorithms provide practical bioinformatics solutions that could be seamlessly integrated into existing bioinformatics pipelines, and to facilitate automated auditing of shared genomic data.

The disclosed watermarking algorithms and methods may be used to provide full control over genomic data to the data owner by enabling traceability and auditability of data access and usage as preferred or agreed upon by the data owner. These algorithms may provide for i) a reduction in the cost of implementing and maintaining a dynamic consent platform because of the distributed nature of ownership-based governance, ii) a promotion and facilitation of genomic data sharing, iii) a support of “consent revocation”, and iv) a minimization of the “data holders'” liability from improper handling of the participants' data and the inability to honor the decisions of the participants thoroughly and in real-time. In this way, the disclosed features provide technical solutions to achieve principles of the above-described dynamic consent and ownership-based governance models, as well as other enhanced user controls regarding access to data, usage of data, and tracking/auditing of data.

Example innovations described in the disclosure are the novel use of digital watermarking to enable the tracking and auditing of distributed data. The data is watermarked with selected watermarking elements (e.g., values of data, such as a selected alternate genomic base replacing a reference base determined by a sequence read) at selected locations in a file that are determined using a random seed that is based on a secret key. In some examples, the watermarking innovations may be combined, in full or in part, with the encryption/decryption innovations, disclosed in the above-referenced '575 disclosure and '830 disclosure to provide further control over the genomic data.

Achieving a trustworthy genomic data sharing is imperative if the benefits anticipated from large-scale data sharing are to be realized. The algorithms, methods, and systems described herein enable true ownership-based governance of genomic sequencing data and greatly simplify the attempts to implement dynamic patient consent for biomedical studies. Using the described mechanisms, the data owner will be able to specify and revoke authorizations for data access and use. Such owner-centered data management will improve the trust relationship between the data owner and the data users, removing the barriers for genomic data sharing. Furthermore, with greatly simplified ownership-based governance of the genomic sequencing data, the owner, instead of large diagnostic or healthcare companies that generate and hence control the genomic sequencing data, could potentially benefit financially from sharing his or her own data, as it should be. Furthermore, it is to be understood that genomic data is provided herein as an example, and the disclosed systems and methods may be applied in additional or alternative examples to dynamically encrypt and/or decrypt any suitable data or file type.

The foregoing and other objects, features, and advantages of the invention will become more apparent from the following detailed description, which proceeds with reference to the accompanying figures.

### BRIEF DESCRIPTION OF THE DRAWINGS

FIG. 1 shows an example representation of an allele frequency range split into intervals.

FIG. 2A shows an example plot of results of a Monte Carlo simulations of the expected percentages of watermark bases that can be identified using random secret keys for watermarks of different sizes.

FIG. 2B shows an example plot of results of a Monte Carlo simulation of the percentages of watermark elements that can be identified when the BAM file was merged with another BAM file using random secret keys.

FIG. 3 shows an example Venn diagram of sets of watermark elements received by two entities.

FIG. 4 shows an example Venn diagram of watermark positions in three example BAM files.

FIG. 5 shows an example plot of percentages of common watermark elements remaining after sharing data multiple times.

FIG. 6 shows an example plot of results of a Monte Carlo simulation, showing the expected percentage of watermark elements that can be identified by chance.

FIG. 7 shows example sample genotype data.

FIG. 8 shows an example plot of results of a Monte Carlo simulation, showing the expected percentage of watermark elements that can be identified by chance for a two-digit allele frequency attack.

FIG. 9 shows an example plot of results of a Monte Carlo simulation, showing the expected percentage of watermark elements that can be identified by chance for a one-digit allele frequency attack.

FIG. 10A shows an example visualization of a BAM file via IGV, showing the existence of variant bases as the background noise of sequencing errors.

FIG. 10B shows an example visualization including a spiked-in watermark in the BAM file of FIG. 10A.

FIG. 10C shows possible outcomes at a watermark position when a reference base C is switched to an alternative base A.

FIG. 11 shows an example method for generating a pool of possible watermark elements.

FIG. 12A shows an example visualization of a BAM file via IGV, showing the existence of variant bases as the background noise of sequencing errors.

FIG. 12B shows an example visualization including a spiked-in watermark in the BAM file of FIG. 12A.

FIG. 12C shows possible outcomes at a watermark position when a reference base C is switched to an alternative base A.

FIG. 13A schematically shows a representation of a continuous variable between 0 degrees and 360 degrees that is quantized.

FIG. 13B schematically shows a representation of embedding information into sequences of quantizer indices.

FIG. 14 shows a representation of an allele frequency range being divided and assigned quantizers.

FIG. 15 shows a representation of an allele frequency range being divided and assigned quantizer bins to preserve genotype.

FIG. 16 schematically shows an example of securely mapping genomic positions to a whole genome keystream.

FIG. 17 schematically shows an example of mapping variants to quantizer resolution and index pseudo-random sequences.

FIG. 18 shows an example of sample genotype data that is generated by a few selected variant callers.

### DETAILED DESCRIPTION

The disclosure provides mechanisms that may be used individually and/or collaboratively in any combination to increase a data owner's control over the sharing of data. Digital watermarking is a technique of hiding a message within a noise-tolerant signal. The hidden message can act as a digital fingerprint that can be employed to identify ownership and to monitor usage of protected data. Digital watermarking has gained a lot of attention in recent decades, in particular for copyright protection of multimedia content. Other watermarking application include, but are not limited to, source tracking, broadcast monitoring, content managing and authentication. The challenge in watermarking of genomic data is that there is less uncertainty in the data, and therefore less bandwidth to carry the fingerprint. Furthermore, in contrast to media content, small alterations may reduce significantly the quality of genomic data. To embed a robust watermark into genomic data while preserving data utility, one must consider carefully the properties of the data.

A watermarking scheme applied to genomic data, or any data, may aim to ensure detectability, data utility preservation, robustness, and traceability. Detectability means that it should be possible (e.g., for a data owner with access to an algorithm or other mechanism associated with the application of the watermark) to discover the watermark in a file, or even a portion of a file, with a high degree of confidence, for example, to detect an unauthorized sharing of a data set. Data utility preservation means that the quality of the shared genomic data is not reduced as a result of watermarking, and that the watermarked data does not lead to erroneous scientific conclusions. A watermarking scheme is robust if it is very difficult or impossible to identify and remove the watermark for unauthorized use. A robust watermark scheme should offer strong resistance to collusion attacks, attempting to identify and remove the watermarks by comparing multiple copies of the same data set, each with a its own watermark. Last, traceability is the ability to identify the parties responsible for unauthorized sharing of the data with a high probability.

As will be described in more detail below, the mechanisms may include dynamic watermarking of all or a part of a file storing the data (e.g., to track and/or provide an auditing trail for identifying unauthorized use/distribution of the data and/or to verify the data). As used herein, watermarking may refer to digital watermarking, or the embedding of a marker within noise (e.g., sequencing errors, in the case of genomic data) of a data file, whereby the digital watermark is only perceptible under certain conditions (e.g., after applying an algorithm) and otherwise does not have a perceptible effect on the data quality and data integrity. While these mechanisms may be used for the protection of genomic data, as described in some of the examples below, it is to be understood that the mechanisms may also be applied to other types of data in other formats.

Using genomic data as an example, standard genomic data file formats are Binary Alignment Map (BAM) format for storing genomic sequences and Variant Call Format (VCF) for storing genomic variations. Typically, a bioinformatics pipeline starts with unaligned genomic sequences in FASTQ format, or raw data, which are then aligned and stored in a BAM file. Subsequently, variations present in a BAM file, defined as differences as compared to a reference genome, are determined and recorded in a VCF file. In many cases, VCF files are shared without the corresponding BAM files, or converted into lists of variants that may be loaded into databases or stored as flat files and excel tables.

This disclosure presents example solutions to watermark BAM and VCF files (or lists of variants). A shared VCF file can always be traced back to the BAM file if one is available. Furthermore, watermarking a VCF file serves no purpose if the corresponding BAM file is shared as well, since a new VCF file can be generated from the BAM with a variant caller. In some examples, the disclosed watermarking algorithm to protect VCF files and lists of variants may be used only if variants are shared without the corresponding BAM files.

Auditing of access to shared genomic data as well as prevention of unauthorized usage of data has gained some attention recently, but no workable solutions have been suggested. In one example, a reversible watermarking scheme for BAM files may be used, in which the watermark is hidden in the modifications of soft clips. Soft clip is a part of the read that is not aligned. An aligner may able to align a portion of a read, but clip off the left end or the right end of the read, or both ends, because the read bases do not match the neighboring reference sequence. The watermark soft clip modifications depend on the content of a BAM file and on a secret key. A small portion of the reads is affected, and modifications are added only if they can be reversed using the secret key. The number of soft clip bases modified and the new content of these bases are selected randomly.

Soft clips are typically ignored by variant callers, and the called variants will not be affected by the alterations. At the same time, soft clips are used to detect structural variations and fusions, since a portion of a read may align to the reference sequence elsewhere. Furthermore, one may want to realign a BAM file, for example, with a different aligner or using a new improved reference sequence. With the above approach, approach it is possible to reverse the changes and process the original unwatermarked BAM. The watermarked file, however, cannot be used for a number of important applications without sacrificing data utility.

In many ways, it is more challenging to watermark VCF files and lists of variants than BAM files. Since a VCF file is a compact representation of sequencing data, there is a lot less data to work with, and the data quality reduction is even more of an issue. A variant can be defined as a tuple of genomic position, reference base (REF), or bases in the case of an insertion or a deletion, and alternative allele (ALT). Typically, some categorical and numerical data is available for a variant; at the very least the variant genotype is given.

An example watermarking scheme for variant data may include an approach in which a watermark is embedded into modified genotypes. Changing a variant genotype is a significant alteration that may render watermarked variants useless, or worse, lead to erroneous clinical and biological interpretations. For example, when the genotype is switched to a homozygous reference, the variant is essentially removed and the sample is mistakenly viewed as not having any variant at this position. When the genotype is toggled between heterozygous and homozygous states, the variant utility for clinical applications, or the validity of a clinical interpretation, is significantly reduced. Thus, such an approach applies watermarking only to a small portion of the variants, and estimates watermarked data utility as a percentage of the unwatermarked variants. This minimizes but does not avoid the detrimental effect on data utility.

When watermarked data is shared multiple times, colluding entities may compare the protected objects and detect differences between them. An example general solution includes an approach in which watermarks are constructed in such a way that they are robust against collusion. The examples described above may employ optimization schemes for selection of watermark elements given to multiple entities, that reduce the probability of colluding parties identifying the watermarks.

To clarify the terminology used herein, the following definitions are introduced:
watermark element is one encoded bit of information, the specific element can be either present or absent in the protected object
watermark pool P, is the full set of all potential watermark elements, |P|=Nₚ, where Nₚ is the number of watermark elements in the pool
watermark W, is the set of watermark elements embedded in the protected object, where W ⊆P
watermark discovery is the rejection of the null hypothesis that the full or partial watermark is found in the tested object by chance.

To watermark BAM files in one example, single-base alterations spread uniformly across the entire genome are employed, with introduced changes hidden below the inherent noise present in the data. A watermark element is present at a genomic position, if a specific ALT base (different from the reference base) is found only in one read at this position. This definition can be generalized to a specific number of reads with the target ALT.

For variant watermarking, Quantization Index Modulation (QIM) applied to some rational data associated with the variants is utilized. In most cases, the watermark will be hidden in Variant Allele Frequency (AF), which may be present in variant data implicitly, but other continuous variables may be used. The watermark element is present if the AF (or another watermark variable) value is assigned to the target quantizer.

While watermark elements inserted into sequencing and variation data are different, the disclosure includes a single approach to both BAM and VCF watermarking:
Watermark elements are small modifications made pseudo-randomly in the protected file that are detectible with a secret key. The watermarking algorithm guarantees robustness by relying upon a secret key, making watermarking discovery prohibitively expensive.
The alterations do not significantly affect the data quality. As a result of this, long watermarks that span the whole BAM and VCF files or variants lists can be embedded.
Watermarks are not reversible. The protected data is reliable, and can used in place of the original data, without affecting data processing and interpretation.
Each time the data is shared, watermark elements can be selected from a larger pool of all watermark elements to embed specific access control policies into data, and to protect against collusion attacks.
Only high quality data points are used for watermarking. The unmodified data can be separated into three groups: data points that are not usable for watermarking, data points that contain a watermark element by chance, and data points that can potentially be used as watermark elements.

### BAM Data Utility Model

The extent of the added alterations should be several orders of magnitude smaller than the background noise due to sequencing errors. In addition to that, most of the modified reads and the corresponding mate pairs should align the same way if realigned. Small changes in mapping quality of modified reads are tolerated, but very few new insertion and deletions (indels), altered soft clips, different read start and end positions, as well as additional split reads should be introduced.

### Variant Data Utility Model

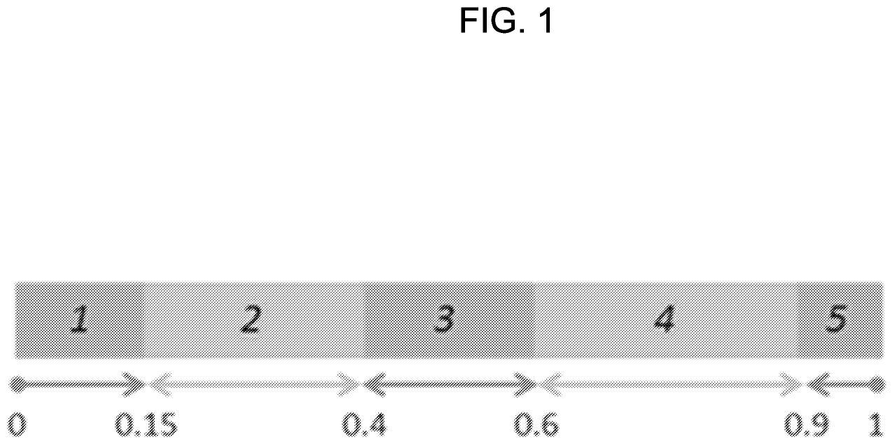

To quantify the effect of AF modifications on data quality, a genotype preservation model is introduced. The range of AF, [0,1], is split into five intervals: three correspond to a specific genotype or a type of a variant, two—to undefined genotype states (FIG. 1).

As shown in FIG. 1, an example AF range is split into five intervals based on genotype states, indexed {1, . . . ,5}. The genotype is preserved when the altered AF stays within the same interval or is moved to the adjacent interval, but does not jump over an interval.

The five intervals are indexed, {1, . . . , 5}, and each AF value corresponds to a specific interval index, idx(AF).
1. 0≤AF≤0.15—somatic variant or homozygous reference
2. 0.15<AF<0.4—undefined
3. 0.4≤AF≤0.6—heterozygous variant
4. 0.6<AF<0.9—undefined
5. 0.9≤AF≤1—homozygous variant
   Definition: The genotype is preserved under a modification of variant allele frequency, if the change in the corresponding interval indices is less than or equal to one: |idx(AF_{new})−idx(AF_{original})|≤1.

Note that, while zygosity (being either homozygous or heterozygous) has little biological relevance when discussing a somatic variant found in a tumor sample, the described data utility definition protects against extreme changes of AF.

### Results

The following two watermarking methods are implemented for genomic sequencing and variation data, which provide ownership protection, enable traceability and audit control, and act as a deterrence mechanism to prevent unauthorized sharing and usage of genomic data.

### Watermarking BAM files

A relatively dense watermark, with one watermark element per 1,000 bases, translates into about 35,000 watermark elements for a Whole Exome Sequencing (WES) BAM file (˜170× depth of coverage), and 300—for a high-depth of coverage (5,000×) panel BAM. The typical percentages of positions that fall into different categories are given in Table 1 (depth threshold was set at 50×).

TABLE 1
Four categories of potential watermarking positions
for two typical types of BAM files.

| | CES | Panel |
|---|---|---|
| Watermark position | BAM | BAM |
| Rejected because of insufficient depth | 6% | 1% |
| of coverage | | |
| Rejected because of multiple reads | 2% | 55% |
| with ALT | | |
| Watermark element present by change: | 11% | 14% |
| exactly one read with ALT | | |
| Can be used for watermarking: no reads | 81% | 30% |
| with ALT | | |

In some cases, when data has a high error rate and/or a high depth of coverage, the number of watermark elements present in data by chance may be too large to rely on a single read containing the alternative watermark base at a watermark position. To further separate the number of positions at which the data can be modified from the number of watermark elements present in the file by chance, the watermark elements can be comprised of multiple reads with the same alternative watermark base at the watermark position (in practice, 2 or 3 reads may be used). The watermark element positions will then contain the exact (threshold) number of the reads with the alternative watermark base, while at positions useable for watermarking the number of reads with the ALT base will be below the threshold. This generalized watermarking approach will result in more reads being modified. However, since it will be applied to data with a high degree of base variation, the data utility will not be affected.

To preserve data utility, low base qualities are assigned to the modified bases. Before inserting the watermark ALT bases, the entire BAM file is surveyed to select the most appropriate quality score to assign to the modified bases. Depending on the data, either the most common low quality base score is assigned, or the score is selected from the set of low quality scores, based on their frequency in the BAM file.

The ‘one element per 1,000 bases’ watermarking results in only 0.02% of all WES BAM reads being modified at a single base. For the high-depth panel BAM, the percentage is even lower: 0.002%. The watermarking scheme preserves data quality, since these percentages are well below the sequencing errors of even the best sequencing technologies available, and the low-quality bases changes are expected to have an even lesser effect. In the following WES tests, the watermark elements were selected randomly from the pool with the probability of p=0.8, which resulted in about 28,000 elements.

The watermarking scheme supports detectability. Given the secret key that was used to generate the watermark, 100% of all watermark elements are recovered if the BAM file has not been further modified. In contrast, only 12% of all watermarks are uncovered for a typical WES BAM by chance, 30%—for a high-depth targeted resequencing BAM.

The watermarking scheme is robust across a wide range of watermark sizes (the number of watermark elements). Specifically, when as few as 100 watermark elements are used, 9.7% of watermark elements are discovered by chance (WES BAM, Table 2A). This is in comparison to 11.8% when 28,134 watermark positions are used, and the percentage remains relatively unchanged across different watermark sizes. By comparison, the algorithm can identify 100% of all watermarks checked with the secret key, from as few as 100 to as many as 28,134 watermark positions in a typical WES BAM file (Table 2B).

TABLE 2
Strong protection with the watermarking scheme when a wide range of numbers of
watermark positions are tested, exemplified with a typical WES BAM file. Columns
“low depth”, “multiple alts”, “alt discovered” and “not discovered” are the categories
of positions encountered when a BAM file is surveyed for potential watermarking
positions. The “% discovered” column lists the percentage of watermark elements
that can be identified when A. random seed is used to generate the watermark,
B. the correct secret key is used.
A. Random Seed:

| Watermark | Low | Multiple | Alt | Not | % |
|---|---|---|---|---|---|
| size | depth | alts | discovered | discovered | discovered |
| 28134 | 5.5% | 2.9% | 10.8% | 80.8% | 11.8% |
| 10000 | 5.6% | 3% | 11% | 80.4% | 12.1% |
| 5000 | 5.8% | 2.9% | 11.5% | 79.8% | 12.5% |
| 1000 | 4.9% | 2% | 10.2% | 82.9% | 11% |
| 500 | 5.6% | 2.4% | 12.2% | 79.8% | 13.3% |
| 100 | 3% | 4% | 9% | 84% | 9.7% |

B. Secret key:

| Watermark | Low | Multiple | Alt | Not | % |
|---|---|---|---|---|---|
| size | depth | alts | discovered | discovered | discovered |
| 28134 | 5.5% | 3% | 91.5% | 0% | 100% |
| 10000 | 5.4% | 3.2% | 91.4% | 0% | 100% |
| 5000 | 5.3% | 2.9% | 91.8% | 0% | 100% |
| 1000 | 5.6% | 2.3% | 92.1% | 0% | 100% |
| 500 | 6% | 3.6% | 90.4% | 0% | 100% |
| 100 | 5% | 3% | 92% | 0% | 100% |

Therefore, a watermark of a sufficient size can protect even relatively small genomic regions, extracted from the BAM file.

### BAM Watermark Discovery

In some examples, a Monte Carlo simulation is employed to estimate the percentage of watermark elements that can be discovered by chance in a target BAM file. The results are then compared to the percentage of elements discovered using the specific secret key. Random watermarks of the same size are generated repeatedly with arbitrary seeds, and the percentage of discovered watermark elements is determined. Given enough Monte-Carlo iterations, the sample mean and the standard deviation can be estimated, as well as the Z-score and the p-value of discovering the specific watermark by chance.

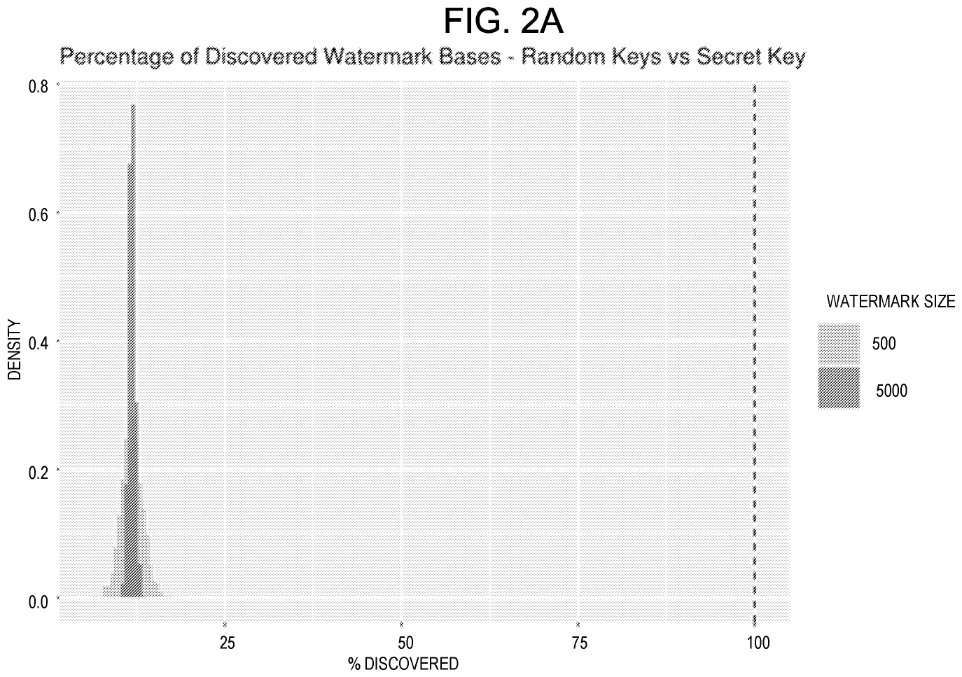

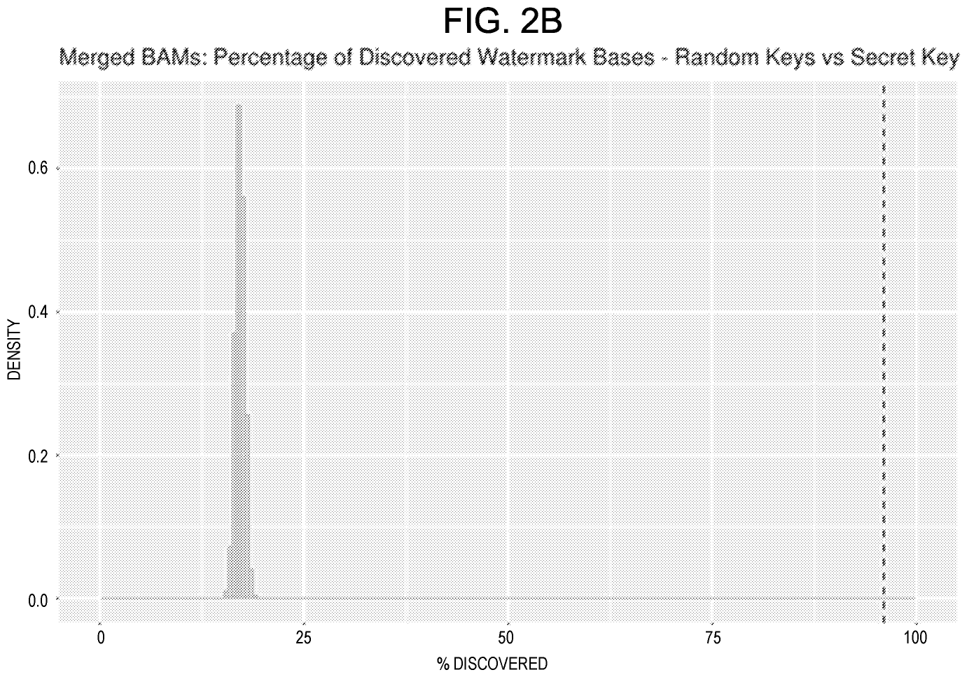

FIG. 2A shows Monte Carlo simulation of the expected percentage of watermark elements that can be identified by chance, for watermark of different sizes. FIG. 2B shows a Monte Carlo simulation of identified watermark elements when the BAM file is merged with another BAM file (watermark size: 5000 elements). In FIGS. 2A and 2B, the dotted line represents the percentage of watermark elements found with the secret key.

For efficiency, the simulation was done with sparse watermarks. FIG. 2A illustrates the results of the Monte Carlo simulations with watermarks down sampled to 5,000 and 500 elements, respectively; 1,000 iterations were performed. For watermarks of size 5,000, the percentage of the watermark elements discovered by chance seed is typically less than 12%. In fact, the 95% confidence interval of the mean falls between 11.80-11.86%. As stated before, the algorithm could identify 100% of watermark elements when the master seed was used, which is 191 standard deviations away from the mean. In other words, the probability of revealing the embedded watermark by chance is close to 0. With only 500 watermark elements, the distribution of the percentage of watermark elements discovered by chance is slightly wider (FIG. 2A). The 95% confidence interval of the mean falls between 11.75-11.93% and the Z-score from the mean is 58 for 100% of all watermarks being discovered with the master seed.

This shows the robustness of the watermarking algorithm, which is further illustrated by another simulation test. In this test, a watermarked WES BAM is merged with a randomly selected WES BAM file of another individual. With the secret key, the analysis can still identify 96% of the watermark elements, while only about 18% of watermark elements are identified by chance in the merged BAM file. With Monte Carlo simulation performed on the merged WES BAM file, as above, the 95% confidence interval of the mean of percentage of watermark elements discovered with an arbitrary seed falls between 17.13-17.20%, while the percentage of the watermark elements discovered with the secret key is 96%, which is 151 standard deviations away from the mean (FIG. 2B).

### Variation Removal Attack

It is possible to completely remove the described BAM watermarks by discarding all single base variation in the pileup. For the example WES BAM file, 31% of all target regions positions are the single ALT bases, and these single ALT bases are found in 10% of the reads. For the example high depth of coverage Panel BAM files, the numbers are: 38% of positions, and 10% of the reads. Removing or modifying 10% of all the reads to eliminate variation, is a very significant alteration of a BAM file. Furthermore, sequencing errors happen randomly, they are scattered throughout, and are often impossible to separate from true biological signals, for example, as in the case of tumor sequencing. Altering all single base variation in a BAM file will result in a drastic loss of data utility.

### Collusion Attack to Infer BAM Watermark

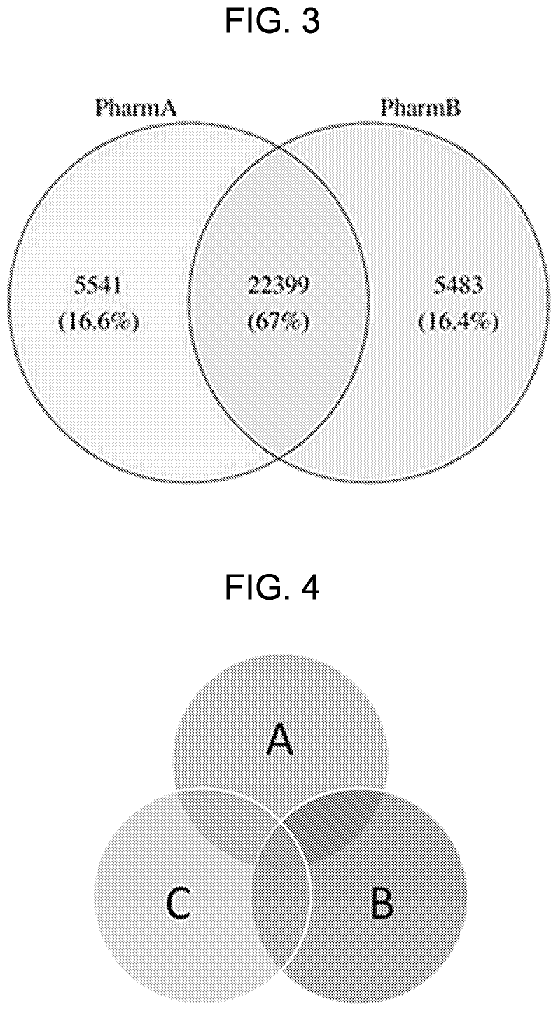

Slightly different subsets of watermark elements are embedded into data to prevent collusion attacks when the same BAM file is shared with multiple entities or with the same entities repeatedly. The common watermark elements protect against watermark inference attacks, when multiple parties collude (FIG. 3).

FIG. 3 shows a Venn diagram of the sets of watermark elements received by two entities, PharmA and PharmB, selected from the pool of watermark elements with probability p=0.8 and entity-specific secret seed. While the two parties can determine the reads different between the BAM files that they have received and remove or modify them, the common watermark elements provide the evidence of the collusion.

FIG. 4 shows a Venn diagram that illustrates the overlapping watermarks of BAM files shared with any two or all three entities A, B, and C. Suppose, entities A and B have colluded together and removed the reads that are different between their BAM files. The dark red section of the Venn diagram will provide the evidence that specifically A and B, but not C, altered the watermark.

The watermarking algorithm supports traceability by identifying parties responsible for the unauthorized sharing with a high probability. The scheme provides strong protection against collusion attacks, when a portion of the data is modified in order to damage the watermark (FIG. 4).

The percentage of common watermark elements decreases with each share, and there are limits of how many times the data can be shared, especially if the data is shared with the same entity, for example, with different policies embedded in the watermarks.

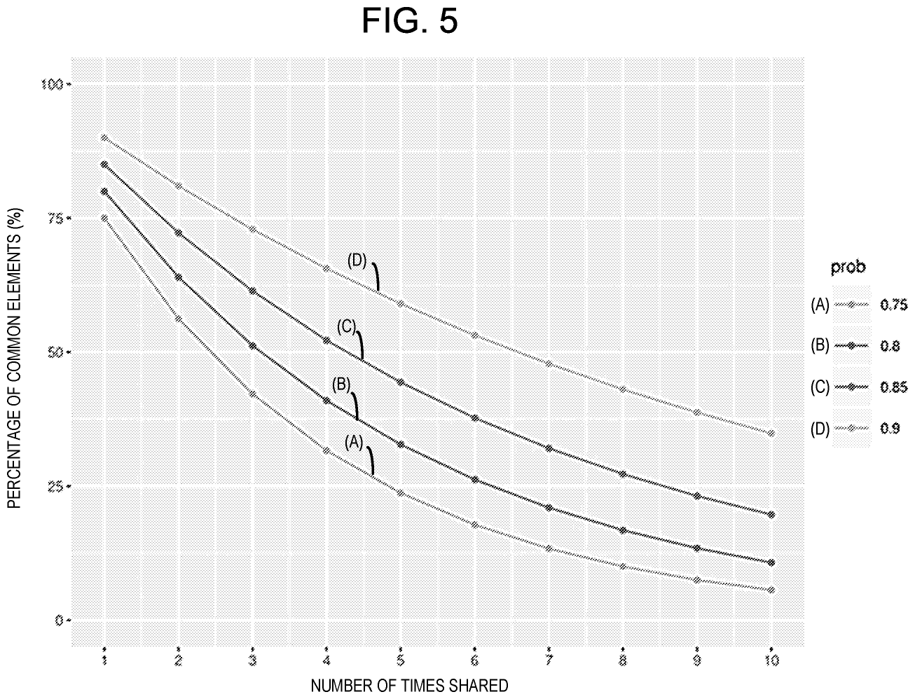

If each watermark element is selected from the watermark element pool independently from other elements with a probability p, after m watermarks are generated, the probability that an element is present in all watermarks is p^{m}. Because of linearity of expectation, the number of common elements after m shares is N_{w}p^{m}, where N_{w} is the size of the watermark elements pool. The percentage of common watermark elements decreases exponentially, but when the base is close to 1, it does not drop below 10-20% after 10 initial shares (FIG. 5).

FIG. 5 shows percentages of common watermark elements remaining after sharing the data multiple times, are presented for different watermark element selection probabilities p={0.75, 0.8, 0.85, 0.9}.

If a BAM file is shared 10 times with the same entity, and p=0.8, the watermark will be reduced to 10.74% of the full set. For the test watermark with 28,000 elements that will result in ˜3,000 undiscoverable elements. If variants are selected with the probability p=0.75, that number of undiscoverable elements will be reduced to ˜1500.

### Realignment of Watermarked BAM Reads

Modified reads that contain watermark elements and their corresponding mates (in paired-end sequencing), in some cases, may align differently than the original reads. This is especially likely if the aligner had problems with the alignment of the unmodified read, and had to add soft clips or assign a very low mapping quality to it. Paired reads are aligned together with their mate reads, so both modified reads and the corresponding unmodified mate pairs may be affected.

To test BAM data utility preservation, the watermarked WES BAM (watermark size=31632) may be realigned with BWA-MEM, the same aligner that was used to produce the original file. 50566 modified reads and unmodified mate pairs were compared between the original watermarked and the realigned watermarked files. Mismatching read positions, cigars, mapping qualities, insert sizes and new split reads accounted for 1,259 conflicts between the original and realigned BAM files, out of which only 191 were due to different mapping qualities (acceptable changes).

The number of unacceptable changes was significantly reduced by selecting only high quality reads for watermarking:
mapping quality is greater than zero
the cigar is not empty and the read is not clipped
if a read is paired: the insert size is less than 600 bp, mate alignment should be on the same chromosome

With this read selection procedure, the number of conflicts is reduced to 227, out of which 174 were changes in mapping quality only. Different cigars, insert sizes or newly introduced split reads accounted for 53 conflicts, or 0.1% of watermark reads and their mate pairs.

### Variant Watermark Discovery

The automated watermark discovery was tested and a number of possible attacks and modifications of a watermark using a whole-exome sequencing (WES) VCF file were simulated, with variants called at or above 2% AF. When the minimum depth of coverage cutoff was set at 100×, 76 variants with sufficient coverage were available for watermarking. The structure of the VCF file was not relied upon and the order of the variants did not matter either. Therefore, the results would have been the same if an unordered list of variants or an unordered VCF file was tested. The order in which the variants were processed, i.e., the specific index of a variant in the generated N and I sequences, was saved in the hash values file, however, the variants themselves did not need to be ordered. The quantizer resolution discrete random variable N was uniformly distributed between 4 and 50, the binomial quantizer index I was generated with the probability of 0.5.

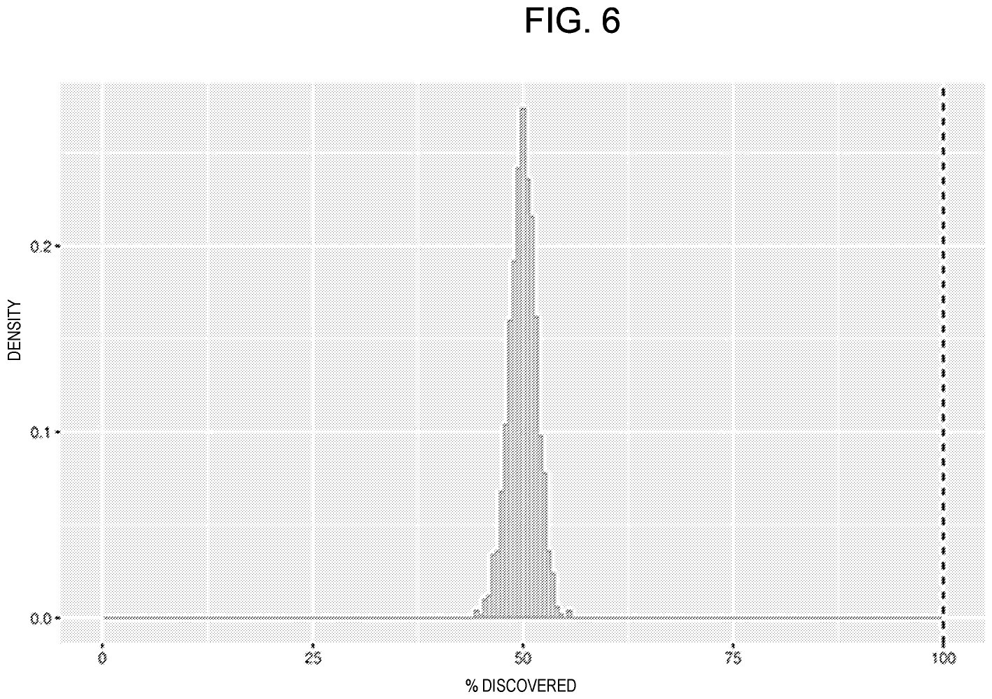

Similarly to the BAM watermark discovery procedure, Monte Carlo Simulation was employed to estimate the percentage of watermark elements (matching quantizer indices) found in the test VCF file by chance. Random sequences N and I were generated, and the test variant quantizer index was checked against the expected index. The procedure was repeated 1,000 times to estimate the mean and the standard deviation of the percentage of discovered watermark elements. The percentage of watermark elements found with the correct secret key was estimated as well. As expected, all elements were discovered in the unmodified VCF file (FIG. 6).

FIG. 6 shows example Monte Carlo simulation results: the expected percentage of watermark elements that can be identified by chance. Mean is 49.96 (95% confidence interval [49.87, 50.07]), standard deviation—1.60. The dotted line represents the percentage of watermark elements found with the secret key (100%). The Z-score is 31.31, and the probability of discovering all elements by chance is essentially zero.

The quantizers split the range of AF into equal portions. Had AF been uniformly distributed, the number of watermark elements found by chance may be expected to follow the binomial distribution with the probability p=0.5. However, the AF is not uniformly distributed and, in general, is not symmetric within the [0,1] interval. Typically, there are peaks around 0.5 and 1.0, and if low AF cutoff is applied (e.g., for somatic data), there is an additional peak at 0. Nevertheless, most of the applied quantizers are of high resolution, and the AF values are equally distributed between the smaller bin intervals. Therefore, the aggregated distribution of the number of elements discovered by chance is actually very close to binomial: in one example (FIG. 6), the mean is close to 50%, and the standard deviation, σ²=np(1-p), where n is the number of tries, is equal to 0.5*\sqrt{97}6=15.62, or if scaled to the percentage of successes, σ=1.60%, as obtained by the simulation results.

Assuming the binomial distribution with p=0.5, the minimum number of watermark elements needed for the watermark discovery can be estimated. If the variant list has not been modified, and all n watermark elements were discovered, the probability of this happening by chance is pⁿ. Therefore, to confidently discover the watermark with p-value<0.05, n should be greater than 4, with p-value<0.01, n>6. This again demonstrates the robustness of the described watermarking scheme, since typically each whole genome sequencing data set contains 3-5 millions of variants, while each whole exome data set −20,000-30,000 variants.

### Subsetting the Watermarked Variants

If a subset of variants is extracted, to split the variant list or to get variants from specific genomic regions (e.g., genes that predispose for a particular disease), the robustness of the watermark will depend on the number of watermark variants present in the subset. As discussed in the previous section, the number of watermark elements should be at least 5, to enable watermark discovery. Since a watermark is employed that spans the whole variant list, and all quality variants are watermarked, the number of remaining watermark elements will be proportional to the size of the extracted regions.

### Variant Addition Attack

Suppose the watermarked VCF variants are merged with multiple other VCF files or variant lists from different samples. This can be done by an attacker who tries to hide a protected VCF file, or a data consumer adding the VCF to a variant storage without a malicious intent. If the variants are unmodified, all watermark elements may be expected to be found, with the exception of common variants present in many biological samples. Table 3 presents the number of unique watermark variants discovered when the protected VCF is merged with 1 to 10 unrelated VCF files. A watermark of a substantial size (615 elements) remains after the protected VCF file is merged with 10 other VCFs.

TABLE 3
Number of watermark elements discovered
after the protected VCF file is merged with
1 to 10 additional unrelated VCF files.

| | Watermark |
|---|---|
| Files | Elements |
| Protected file only | 976 |
| 1 file added | 946 |
| 2 files added | 774 |
| 3 files added | 708 |
| 4 files added | 669 |
| 5 files added | 666 |
| 6 files added | 635 |
| 7 files added | 630 |
| 8 files added | 630 |
| 9 files added | 622 |
| 10 files added | 615 |

### AF Precision Reduction

An attacker may attempt to remove the watermark by dropping a number of less significant digits from the AF value. The AF precision may also be reduced from rounding up the values, without a malicious intent. Additional data that can be used to recover the precise AF values (e.g., AO and DP) may be assumed to be not available, and the rounded-off AF values are used for watermark discovery.

TABLE 4
AF precision reduction simulation results.

| precision | missed | found | Percentage |
|---|---|---|---|
| Full | 0 | 976 | 100% |
| 5 | 0 | 976 | 100% |
| 4 | 0 | 976 | 100% |
| 3 | 84 | 892 | 91.39% |
| 2 | 202 | 774 | 79.30% |
| 1 | 420 | 556 | 56.97% |

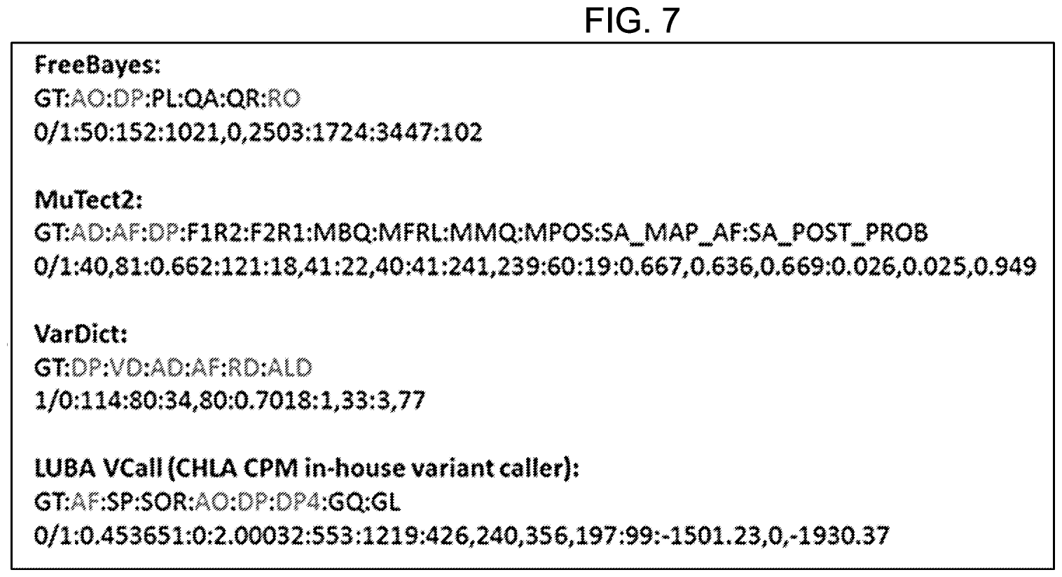

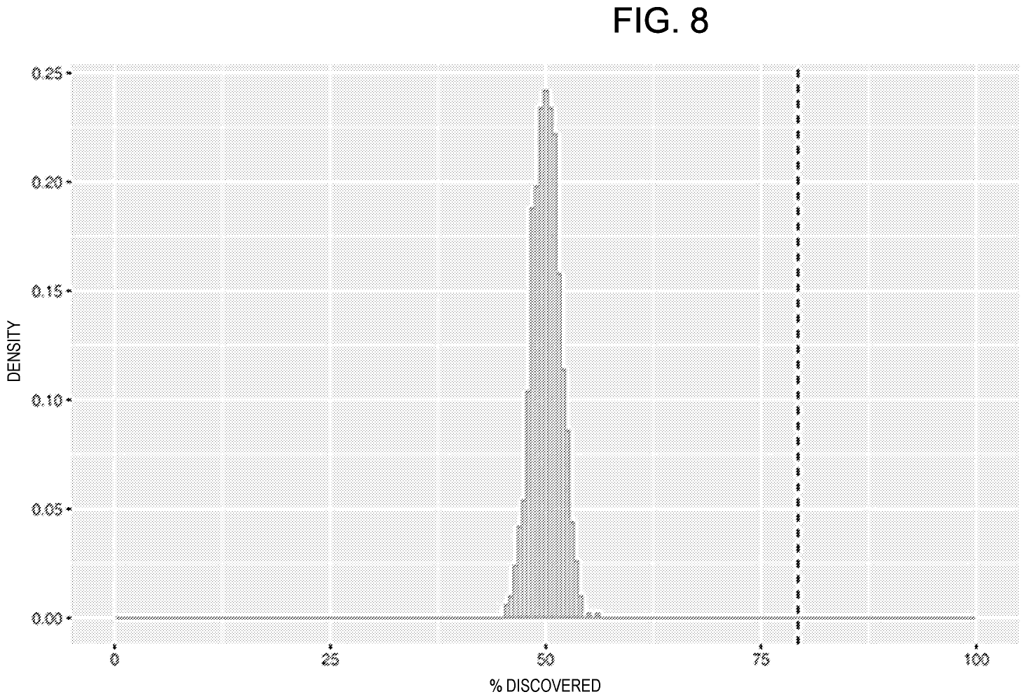

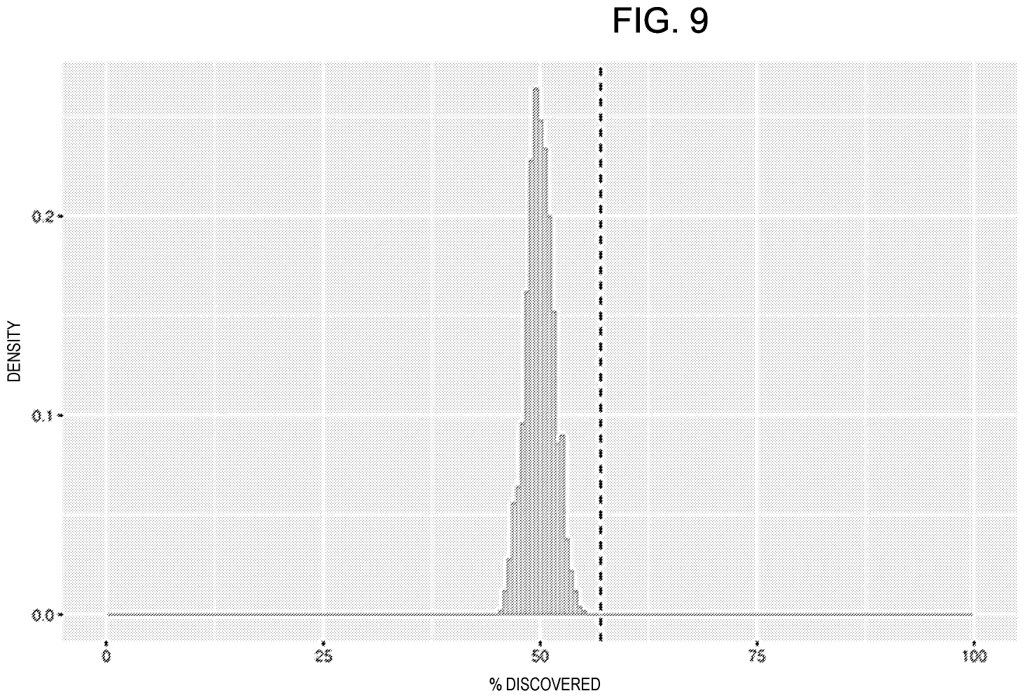

Typically, variant callers report 3 to 6 decimal digits (FIG. 7). In an example test, the full precision is set at 6 decimal digits, and reduced the number of decimal digits to {5, 4, 3, 2, 1}. The removal of all decimal digits is not considered, because when AF is reduced to {0,1} values, the heterozygous genotypes are lost. With the test sample and the choice of quantizers, the reduction of AF resolution to 5 and 4 decimal digits does not affect the quantization. When the number of decimal digits is reduced further, the watermark discovery is affected (Table 4). However, even at 2 digits and 1 digit precision, the percentages of found watermark elements are statistically significant (FIGS. 8 and 9).

FIG. 7 shows sample genotype data generated by selected commonly used variant callers (FreeeBayes, MuTect2, VarDict) and a custom variant caller LUBA. The tags directly related to the AF are highlighted: AO and VD—alternate allele count or variant depth, RO and RD—reference allele count or depth, DP—depth of coverage, ALD and DP4 contain information about forward and reverse strand allele counts.

FIG. 8 shows a two-digit AF precision attack. Monte Carlo simulation results: the expected percentage of watermark elements that can be identified by chance. Mean is 50.05, standard deviation—1.58. The dotted line represents the percentage of watermark elements found with the secret key (79.30%). The Z-score is 18.58, and the probability of finding this percentage of watermark elements by chance is essentially zero.

FIG. 9 shows a one-digit AF precision attack. Monte Carlo simulation results: the expected percentage of watermark elements that can be identified by chance. Mean is 49.96, standard deviation—1.58. The dotted line represents the percentage of watermark elements found with the secret key (56.97%). The Z-score is 4.44, and the p-value is 0.46·10⁻⁶, i.e., the watermark is present in the VCF with a high probability.

In addition to the aggregated percentage of discovered watermark elements, the data of performance of individual quantizers of specific resolutions, N={4, . . . , 50}, is available as well. This makes the watermark even more robust to precision reduction attacks, because of how some quantizers split the [0,1] interval. For example, quantizers with resolution N equal to 50 and 25, with half-bin sizes of 0.01 and 0.02 respectively, are not affected by the precision reduction to 3 and 2 digits. At the same time, the quantizers with resolution N=5, and the half-bin size of 0.1, are not affected by the precision reduction at all (Table 5).

TABLE 5
Utilizing specific quantizer resolution data to counter reduced
AF precision attack. The number of variants quantized with
specific resolution (“size”), and the percentages of discovered
watermark elements at the 1, 2 and 3 digits precision reduction,
are presented for all quantizer resolutions N = {4, . . . , 50}.

| quant | size | 3 digits | 2 digits | 1 digit |
|---|---|---|---|---|
| All | 976 | 91% | 79% | 56% |
| 50 | 17 | 100% | 100% | 41% |
| 49 | 17 | 88% | 64% | 70% |
| 48 | 15 | 100% | 73% | 33% |
| 47 | 29 | 93% | 79% | 55% |
| 46 | 17 | 88% | 76% | 47% |
| 45 | 20 | 90% | 75% | 60% |
| 44 | 17 | 94% | 76% | 64% |
| 43 | 23 | 86% | 60% | 43% |
| 42 | 22 | 77% | 54% | 54% |
| 41 | 15 | 93% | 73% | 53% |
| 40 | 20 | 95% | 60% | 45% |
| 39 | 26 | 96% | 80% | 38% |
| 38 | 18 | 94% | 83% | 61% |
| 37 | 19 | 94% | 84% | 73% |
| 36 | 27 | 85% | 77% | 44% |
| 35 | 22 | 95% | 86% | 45% |
| 34 | 34 | 85% | 67% | 41% |
| 33 | 28 | 89% | 85% | 53% |
| 32 | 18 | 94% | 72% | 72% |
| 31 | 25 | 96% | 76% | 56% |
| 30 | 22 | 95% | 90% | 45% |
| 29 | 21 | 85% | 80% | 57% |
| 28 | 15 | 66% | 66% | 66% |
| 27 | 27 | 92% | 85% | 74% |
| 26 | 29 | 93% | 79% | 62% |
| 25 | 20 | 100% | 100% | 65% |
| 24 | 16 | 68% | 56% | 68% |
| 23 | 29 | 86% | 79% | 62% |
| 22 | 20 | 80% | 65% | 60% |
| 21 | 33 | 96% | 78% | 51% |
| 20 | 22 | 100% | 68% | 50% |
| 19 | 12 | 75% | 66% | 41% |
| 18 | 19 | 84% | 68% | 63% |
| 17 | 20 | 100% | 95% | 50% |
| 16 | 26 | 88% | 57% | 65% |
| 15 | 18 | 94% | 94% | 66% |
| 14 | 16 | 100% | 93% | 62% |
| 13 | 13 | 92% | 92% | 38% |
| 12 | 23 | 86% | 86% | 60% |
| 11 | 23 | 95% | 91% | 47% |
| 10 | 19 | 100% | 100% | 31% |
| 9 | 20 | 95% | 95% | 90% |
| 8 | 14 | 92% | 78% | 42% |
| 7 | 23 | 100% | 91% | 73% |
| 6 | 15 | 80% | 80% | 60% |
| 5 | 21 | 100% | 100% | 100% |
| 4 | 11 | 100% | 81% | 72% |

### Noise Addition Attack

A noise addition attack may be simulated by adding Gaussian noise to the AF: AF_{new}=AF_{original}+ε, where ε= [symbol] (0,σ²). Added distortion is proportional to the variance of the noise, and is independent of the specific AF values.

TABLE 6
Noise addition simulation results.

| | | | GT | GT |
|---|---|---|---|---|
| σ | missed | found | preserved | altered |
| 0.001581 | 191 | 785 | 976 | 0 |
| 0.005 | 283 | 693 | 976 | 0 |
| 0.015811 | 382 | 594 | 976 | 0 |
| 0.05 | 458 | 518 | 976 | 0 |
| 0.1 | 458 | 518 | 972 | 4 |
| 0.158114 | 478 | 498 | 950 | 26 |
| 0.223607 | 500 | 476 | 885 | 91 |
| 0.353553 | 474 | 502 | 761 | 215 |

For smaller values of σ, the altered watermark can be successfully recovered (Table 6). When σ>0.1, the added noise destroys a significant percentage of variant genotypes. When σ=0.1, the genotype is altered for four variants. While, at the 53%, the percentage of discovered watermark elements is low, the watermark can be discovered by utilizing low-resolution quantizers (Table 7).

TABLE 7
Noise addition attack, σ = 0.1.
Low-resolution quantizers
facilitate watermark discovery.

| N | size | Found |
|---|---|---|
| 20 | 335 | 54.03% |
| 19 | 317 | 54.26% |
| 18 | 301 | 54.49% |
| 17 | 287 | 54.01% |
| 16 | 255 | 54.51% |
| 15 | 234 | 55.56% |
| 14 | 213 | 56.34% |
| 13 | 195 | 55.90% |
| 12 | 180 | 57.22% |
| 11 | 161 | 58.39% |
| 10 | 133 | 57.89% |
| 9 | 113 | 57.52% |
| 8 | 96 | 59.38% |
| 7 | 74 | 58.11% |
| 6 | 52 | 61.54% |
| 5 | 26 | 65.38% |

### Collusion Attack on Variant Data

The same approach may be used to protect against collusion attacks, that was described for BAM watermarks.

If the VCF file is shared 10 times with the same entity, and watermark elements are selected with the probability p=0.8, the test watermark with 976 elements that will result in ˜100 undiscoverable elements. If variants are selected with the probability p=0.75, that number of undiscoverable elements will be reduced to ˜50.

### Discussion

In this disclosure, a practical approach to watermarking of genomic data is presented. The described sequencing and variant data watermarking schemes support required watermarking properties: detectability, data utility preservation, robustness, and traceability. The watermark provides ownership protection and audit control, and acts as a deterrence mechanism to prevent unauthorized access.

The described watermarking algorithms work with the standard BAM and VCF formats, and therefore support transparent interoperability with existing genomic pipelines. Watermarking operation is very efficient, and there is negligible overhead in adding a watermark to a file. In fact, it is takes half the time to watermark a WES BAM file as compared to copying it with the SAMTools view command The reason for this is that SAMTools extracts all information from the packed reads to copy them over, while the software just checks the genomic position and the length for most of the reads, and only the reads that are modified are expanded and fully processed.

Since new watermarks are embedded into the data independently from all previous watermarks, this overhead does not increase with subsequent shares. This is in contrast to schemes utilizing optimization techniques to select watermark elements. For example, when some example approaches generate watermarked files with different keys, each added key increases the processing time.

Discovering the watermark is an even faster operation. Multiple Monte Carlo iterations, to repeatedly discover random watermarks and estimate the expected percentage of watermark elements present in a file by chance, are feasible.

The design of the algorithms is guided by the fundamental Kerckhoffs's principle: the security of a cryptographic system or other security mechanism may rely solely on the secrecy of the cryptographic key, not the secrecy of the algorithm. Out implementation is based on robust National Institute of Standards and Technology standardized cryptographic mechanisms: AES-256 and SHA-256, with well-established security properties.

### Data Quality Preservation

Alterations added to a watermarked BAM file are well below the background noise caused by sequencing errors. Furthermore, if a watermarked BAM file is realigned, all but a negligible number of the modified reads and mate pairs will be aligned in the same way as the original reads.

When a VCF file or a list of variants is watermarked, the genotype is preserved, and the majority of variants receive tiny displacements of the variable used for watermarking (e.g., AF). Variant genotypes may be correlated via Linkage Disequilibrium and can be inferred from family data. Therefore, some examples consider a model of correlated data in their watermarking scheme. These concerns are not applicable to the described approach, since the correlation between the data points is not altered.

### Watermark Detectability and Robustness

The described schemes support watermark discovery in partial or modified genomic files. By using a long watermark distributed across the whole data set, the protection of subsetted data may be facilitated. The schemes are robust against the following modifications of watermarked data, which may be caused by attacks or non-malicious transformations:
Protected data set is merged with other data sets. This may be done by a malicious entity trying to hide protected data, or by a data storing entity in the case of variant data. Merging a large number of BAM files may be impractical, but protected variants can be added, for example, to a knowledge base. Watermark discovery in this case will rely on rare variants unique to the watermarked sample.
Variant data—precision reduction. Reduction of precision of AF or other watermark variable will affect the quantization, but the watermark discovery will still be possible, especially, because quantizers of different resolutions are employed.
Variant data—noise addition. Similarly to precision reduction, the AF quantization will be affected. Extreme noise attacks destroy the genotypes, while moderate attacks do not completely break the quantization at lower resolutions, and the watermark discovery is possible.

### Watermark Removal

Clearing or altering all single base variation in a BAM file, to remove the watermark, will result in a significant loss of data utility.

Variant watermark may be removed as well by discarding all watermark data. The AF, used in the described variant watermarking scheme, is very important for interpreting somatic genomic data, since it provides information such as tumor percentage and clonality. For germline data, removal of AF will reduce the quality of the data, but interpretation is unlikely to be affected if the genotype information is intact. If all numerical information is deleted, the disclosed algorithm will still be able to determine the ownership of a list of variants, although policies embedded in the watermark will be lost.

### Watermark Traceability

The disclosed watermarking approach supports traceability, and provides protection against multiple parties colluding together to damage or remove the watermark.

Additional constraints can be added to the watermark, as needed. For example, any attribute of a policy relating to the usage of the data may be incorporated in the additional seed used to select the watermark positions from the pool, in combination with or as an alternative to the entity and/or time validity information described above. The watermarking scheme is dynamic in that watermarks are generated (e.g., at a time of distribution) for particular policies such that the same data may be shared with different entities, at multiple times, and/or repeatedly with the same entity and different policies may be preserved in the data via the different watermarks used each time the data is shared.

Provided a watermark of a substantial size, data with different embedded policies can be shared multiple times, and be protected against a collusion of a relatively large number of colluding parties. The number of times different watermarks can be securely embedded into the same data is limited, but when the data can support a large pool of watermark elements, this limit is large enough for most practical purposes.

The disclosed approach can be applied to short watermarks, for example, when protection of small genomic regions is required. In this case, an optimization scheme similar to some of the above-described example approaches can be utilized for watermark element distribution to multiple parties. In this case, it will not be possible to insert additional information into a watermark. However, since there is more rationale to embed policies into larger data sets, the need for fine grained control of the data is balanced by the amount of data being released.

### Infrastructure for Genomic Privacy Protection

Related concepts may include automated watermark discovery, integration with a blockchain, dynamic encryption.

### Security Built-In

The described BAM file watermarking scheme can be extended to raw sequencing data in FASTQ format generated by a sequencing machine. To watermark this raw data, first, a FASTQ file is aligned to a reference sequence. Next, the aligned BAM file is watermarked. After that, the watermarked BAM file is converted back to the FASTQ format, which results in a watermarked FASTQ file. Subsequently, the original FASTQ file and the intermediate BAM file are destroyed. As demonstrated, almost all of the modified reads and the corresponding mates will align in the same way as the original mate pairs. As a result of this, the watermark discovery will proceed as if the BAM file rather than the FASTQ file was watermarked. Furthermore, this FASTQ watermarking procedure will not significantly reduce the data quality, and the downstream processing will not be affected.

### Methods

### BAM Watermarking

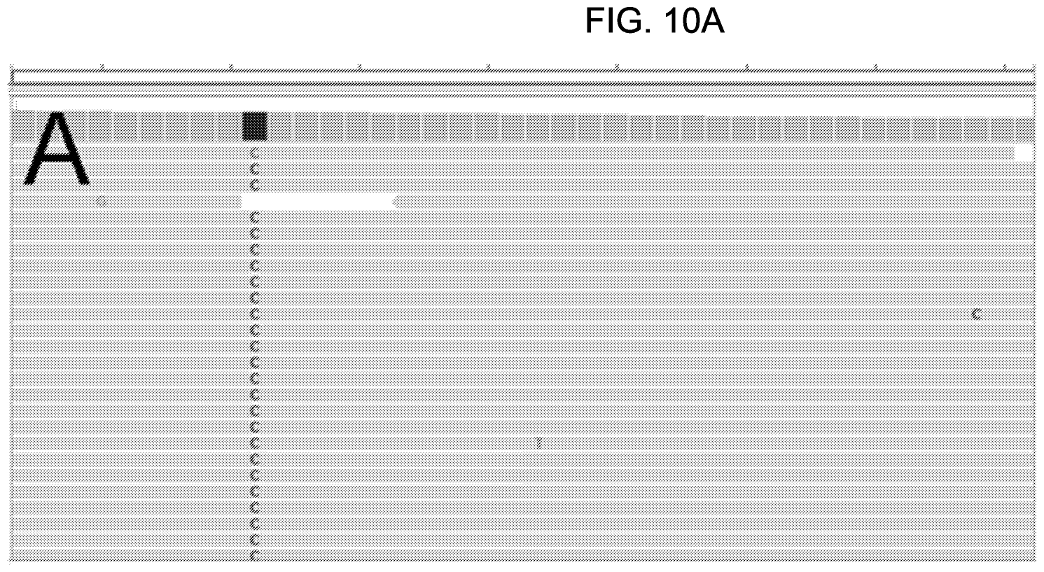

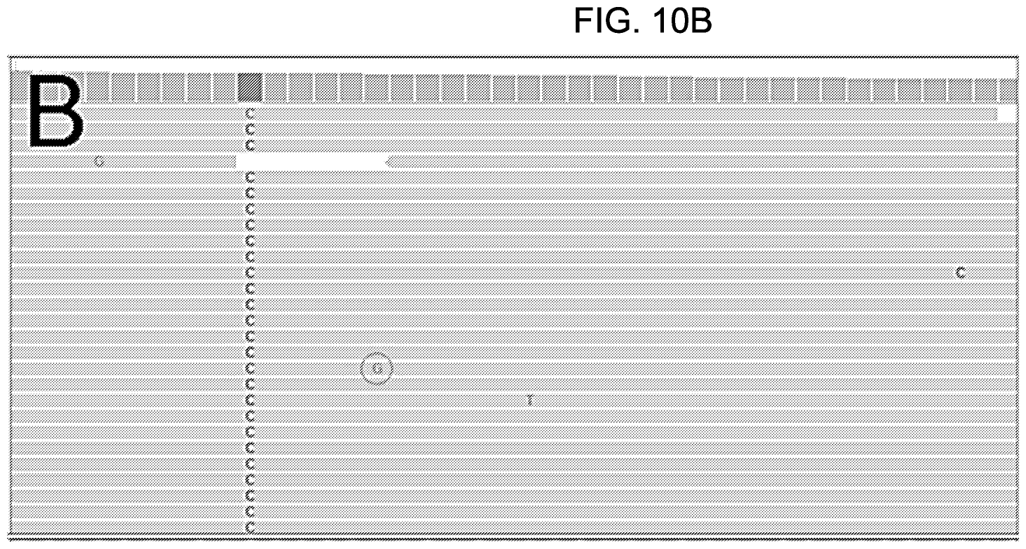

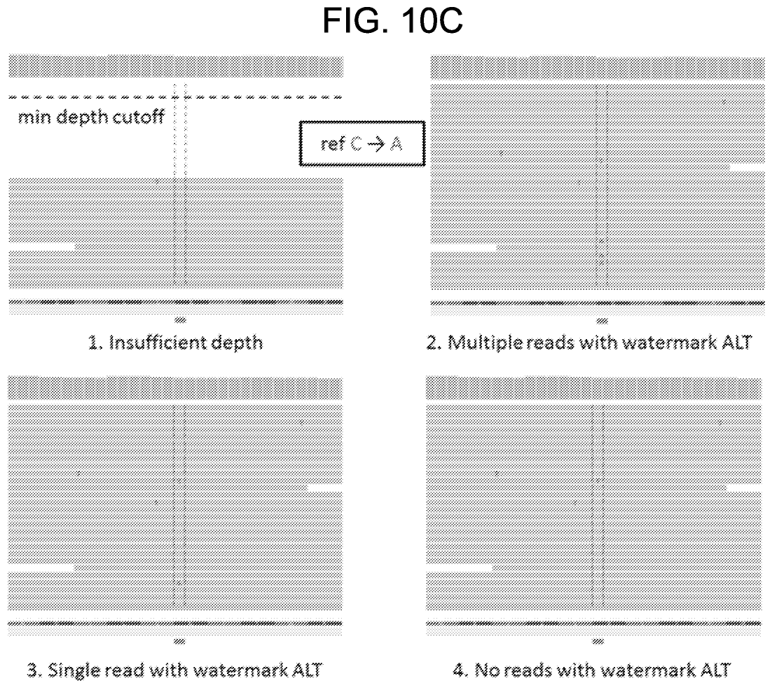

The described BAM watermark is a set of base alterations spread uniformly across the entire genome or across the target regions (FIGS. 10A, 10B). At each watermark position, a base is switched from the reference to one of the three possible alternative bases (e.g., reference base A: A→C, A→G, A→T). Typically, a base is modified in a single sequence read, although in special cases (e.g., BAM files with very high depth of coverage and high base variation) multiple reads may be altered. This modification is done in a deterministic way based on a secret key. As a result of this, the disclosed watermarking algorithm guarantees robustness, making watermark discovery prohibitively expensive. To check whether the watermark is present in a given BAM file, the same sequence of watermark elements (i.e., single specific alternative bases at defined genomic position) is generated based on the secret key, and the percentage of expected watermark elements discovered in the target BAM file is estimated.

FIG. 10A shows an example visualization of a BAM file in the IGV: random variant bases are the sequencing errors. FIG. 10B shows the spiked-in watermark in the same BAM file is circled, indistinguishable from random base variation. FIG. 10C shows four possible outcomes at a watermark position. In this example, reference base C is switched to the alternative base A.

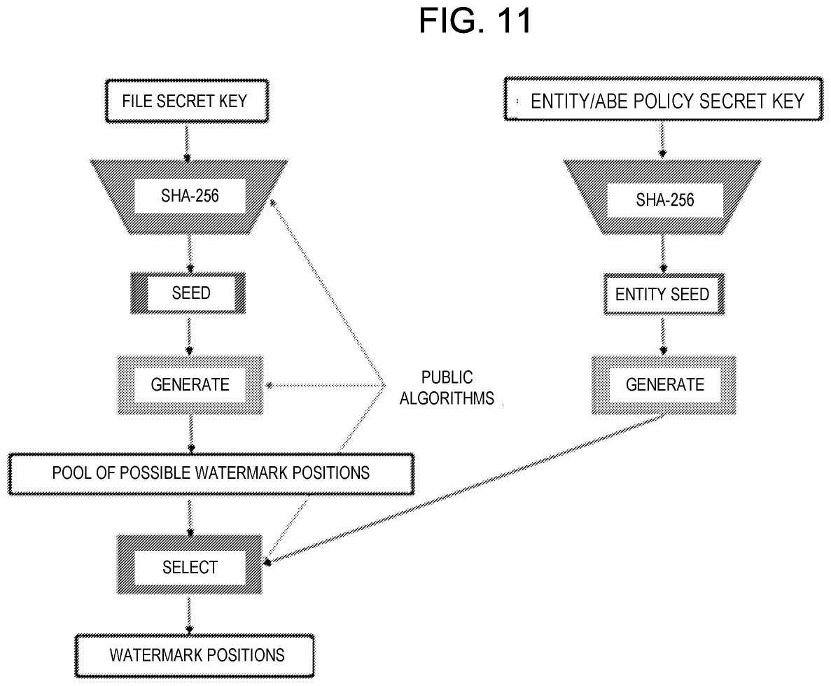

Watermark elements are uniformly distributed across the BAM file with a user-selected density. First the whole genome or target regions are concatenated into a single interval. To select the watermark positions, a random seed (BAM file master seed) is generated with SHA-256 secure hash algorithm, using information derived from the secret key (FIG. 11). Let N_{wv} be the number of watermark elements, N_{wv}=L D_{wv}, where L is the length of the interval, D_{wv} is the watermark density. With the master seed, an ordered pseudorandom set of N_{wv} integers uniformly distributed between 0 and L is generated. Each integer corresponds to a unique genomic position. The generated set is the pool of all possible watermark positions.

FIG. 11 shows an example of how a pool of possible watermark elements is generated with a pseudorandom seed derived from the secret key. The specific watermark elements are selected with an additional random seed, derived from the information about the entity the file is being shared with, and, optionally, user-defined CP-ABE policy

In some examples, ABE policy and/or dynamic encryption may be used.

Next, an additional random seed is generated with SHA-256 from the information about the entity the file is being shared with, and, optionally, the valid time period to access the data, and/or other attributes as desired by the owner and defined in the CP-ABE policy. With the additional seed, the N_{wv} entity- and policy-specific watermark positions are selected from the pool a high probability, e.g., p=0.8. (FIG. 11).

To generate the watermark base modification at each of the possible watermark positions, a pseudorandom integer between 1 and 3 is generated with the master seed. This number defines the transition from the reference base to one of the alternative bases in the ordered set: {A, C, G, T}, which is treated as a circular array. For example, if the reference base is “G” and the transition is 2, the selected ALT base will be “A”.

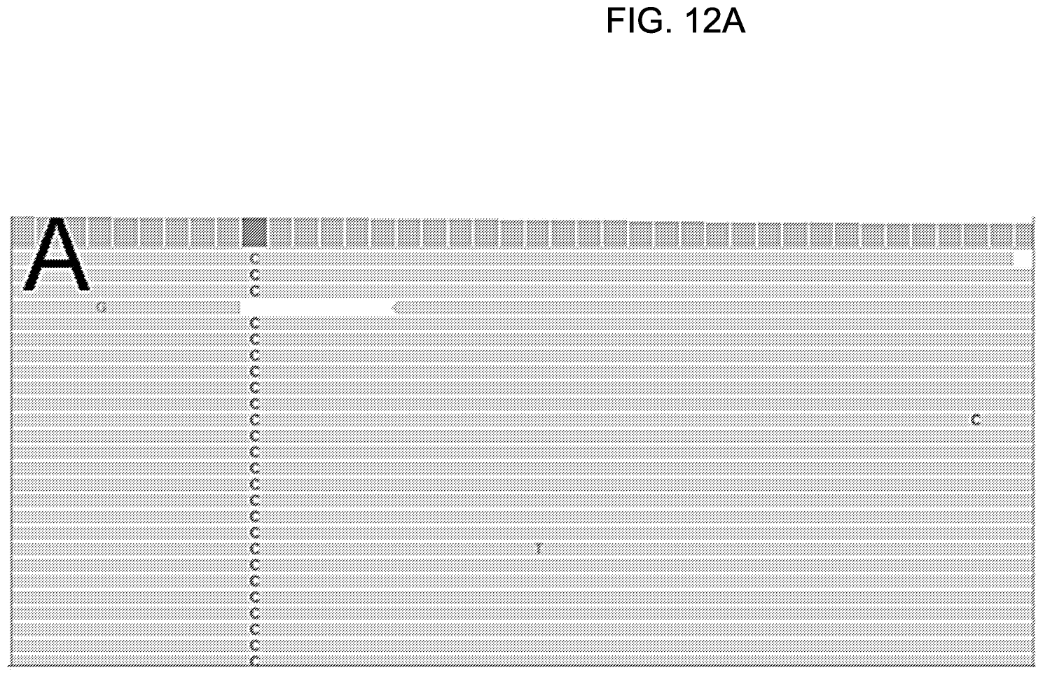

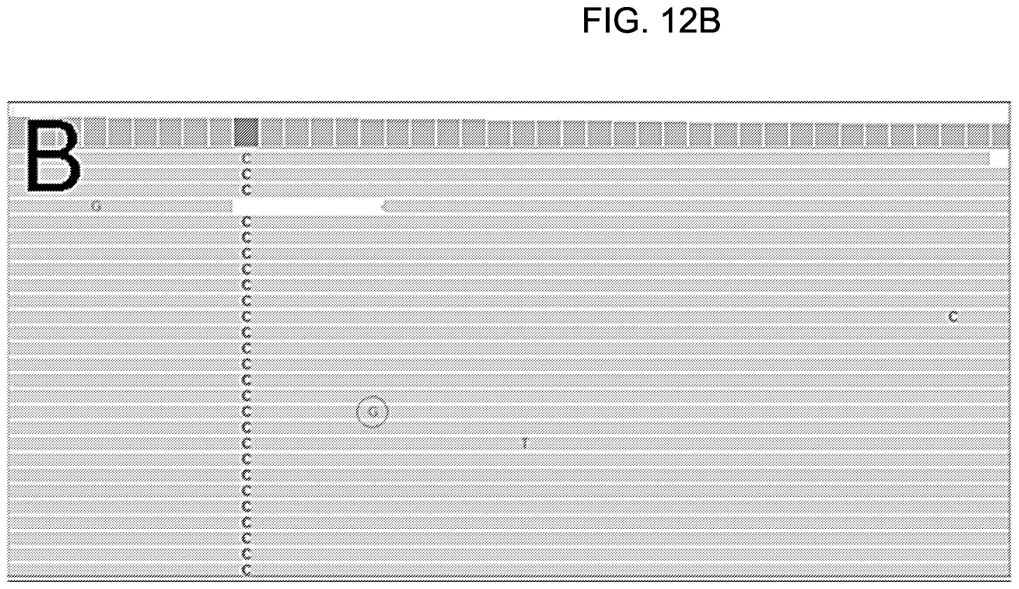

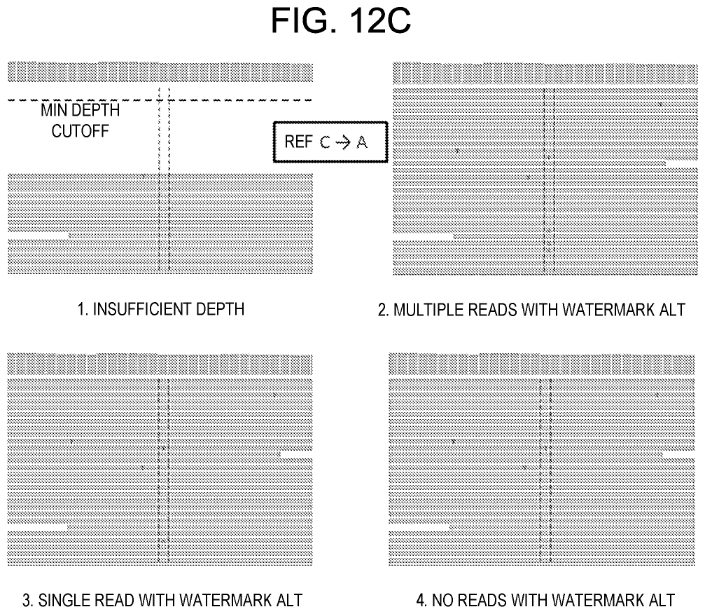

Base alteration is done only if the watermark position meets a certain criteria. The following positions are ignored: positions with insufficient (user-defined) depth of coverage (outcome 1), positions with more than one read with the watermark ALT base (outcome 2), and positions with exactly one read with the watermark ALT (outcome 3) (FIG. 12C). The remaining watermark positions have no reads with watermark ALT base (outcome 4), and are therefore suitable for base alteration. The “outcome 3” positions are essentially the watermark elements present in the BAM file by chance.

FIG. 12A shows an example visualization of a BAM file in the IGV: random variant bases are the sequencing errors. FIG. 12B shows the spiked-in watermark in the same BAM file is circled, indistinguishable from random base variation. FIG. 12C shows four possible outcomes at a watermark position. In this example, reference base C is switched to the alternative base A.

### Variant Data

A variant can be defined as a tuple of genomic position, reference base (REF), or bases in the case of an insertion or a deletion, and alternative allele (ALT). Typically, some variant-associated data, important for variant interpretation, is given with the variant tuple, e.g., genotype, allele frequency, quality of the call, depth of coverage, etc. Watermark can be hidden in small perturbations of the variant data. In most cases, the commonly available Variant Allele Frequency (AF) may be relied upon, but other rational data, for example, variant quality, may be used instead. Some variant callers do not output AF, and instead report other AF-related data. The AF, however, does not need to be present in the variant explicitly. As long as the depth of coverage at the variant position (DP) or the count of reference alleles (RO) is available, along with the alternative alleles count (AO), the watermark can be embedded in the implicitly calculated AF (AF=AO/DP). An aspect of the disclosed method is that the perturbation added to the AF will be small enough, so that it does not affect the genotype call or the quality of the genotype call.

### Quantization Index Modulation

Quantization Index Modulation (QIM) is a notable digital information embedding technique that offers significant advantages over traditional low-bit modulation and spread-spectrum watermarking approaches.

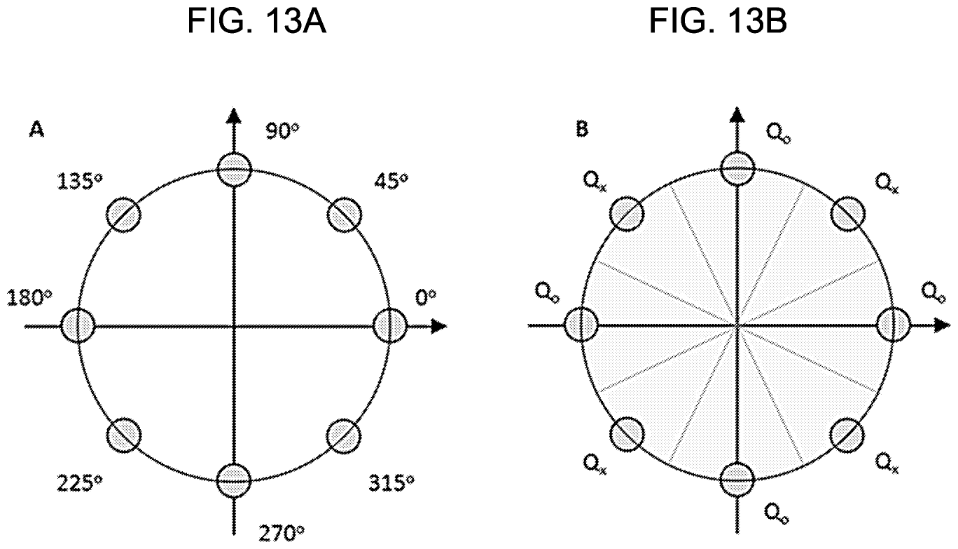

FIG. 13A shows a continuous variable between 0° and 360° (e.g., phase shift) is quantized. FIG. 13B shows when two quantizers are introduced, Qₒ and Qₓ, each data point is mapped to the nearest quantizer. The original signal is perturbed so that data points are assigned to specific quantizers, and the information is embedded into the sequence of quantizer indices (o and x).

Quantizers are discrete approximate identity functions that can map, for example, a continuous variable into a finite set of elements. FIG. 13A demonstrates the approximation of a continuous variable by 8 discrete elements. An ensemble of nonintersecting quantizers can split the space of the approximated variable, so that each data point is assigned to the nearest quantizer. By adding a small perturbation to a data point, one can move it to a specific quantizer, and therefore embed information in the quantizer index. FIG. 13B introduces two sets of four-element quantizers. Using two quantizers, 1 bit of information, {0,1}, can be encoded in the quantizer index {o,x}, and passed along with the perturbed signal.

### Variant Watermarking with QIM

The algorithm is described using AF as an example. AF is a continuous variable that takes values between 0 and 1. The method can be applied to other continuous variables defined on different intervals, as well as to a set of variables.

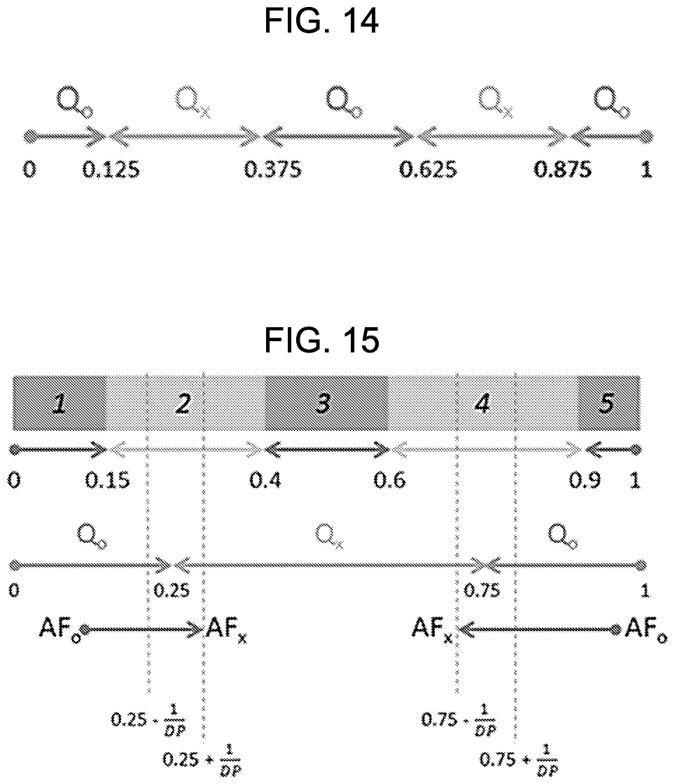

Similarly to the circular example (FIG. 13B), the AF range, [0,1], is divided into N>1 bins of size 1/N, and shift the bins by their half-length, 1/2N, so that the first and the last bins are of size 1/2N. The adjacent bins are assigned to different quantizers, Qₒ and Qₓ, and the bin size defines the resolution of the two quantizers (FIG. 14). Specifically, the half-bin size 1/2N is the maximum displacement needed to move a data point to the dissimilar quantizer interval.

FIG. 14 shows two quantizers with N=4. At each variant position, the AF quantizers bin size, 1/N, and the target quantizer index, O or X, will be selected pseudorandomly, based on a secret key. ALT count (AO) will be adjusted without changing the total depth (DP), so that the corresponding AF falls into the selected quantizer bin. Therefore, the precision of AF displacement is limited by the depth of coverage, DP>N, and the minimum AF change is:

min|ΔAF|=min|ΔAO/DP|=1/DP,

The watermark will be embedded in all variants that have a sufficient depth (e.g., DP>100), if the depth is defined explicitly or can be determined from other parameters. In the case when only the AF data is available without any extra information about the variant, or a different variable is used to embed the watermark, all variants will be watermarked.

Variants with low depth of coverage will not be used as watermark elements, to preserve the genotype. With a minimum depth cutoff for watermark variants, the genotype is preserved at all quantizer resolutions. It needs to be noted that, with continuous drop of sequencing cost, it is expected that the sequencing depth will likely increase to improve data quality. This is especially true with somatic or tumor sequencing tests, including liquid biopsy tests.

When the AF is tied to the AO and DP ratio, the maximum displacement is limited by the half-bin size plus the precision of the ΔAF:

$$( Δ AF ) \leq \frac{1}{2 N} + \frac{1}{DP} .$$

In the described genotype preservation model, the minimum AF displacement needed to jump over an interval is 0.2 (distance between genotype intervals 2 and 4, see FIG. 15. When N=2, the max(ΔAF)>0.2 condition is satisfied for all DP values, but the same is not true for N>2. If, for N>2, the minimum depth of coverage is set at

$${DP}_{min} = \frac{10}{2 - \frac{5}{N}} ,$$

then max(ΔAF)<0.2, and the genotype is preserved. For example, for N=3, DPₘᵢₙ=30, and the minimum required depth of coverage decreases to 5 for higher resolution quantizers.

FIG. 15 shows genotype preservation at the lowest quantizer resolution N=2. Because of the structure of quantizer bins, the altered AF is contained within the target interval if DP≥7.

However, with a minimum depth cutoff, even at the lowest possible quantizer resolution of N=2, the genotype is preserved because of the structure of the quantizer bins (FIG. 15). The altered AF is placed close to the nearest end of the other quantizer bin, with the maximum offset of 1/DP. For example, a somatic variant AFₒ will be moved to the

$$[ 0.25 , 0.25 + \frac{1}{DP} ]$$

interval, and will cross into the heterozygous interval 3, starting at 0.4, only if DP<7. Similarly, if N=2, it is possible to move from the homozygous interval 5 to the heterozygous interval 3, only if DP<7. This shows the robustness of the disclosed watermarking scheme given the continuously decreasing sequencing cost and continuously increasing sequencing depth.

The quantizer resolution N will be varied to balance the robustness of the watermark with the data utility, and to obscure the applied perturbations. As a result of that, a small number of variants will receive a larger perturbation, and will be able to preserve the watermark against more extreme AF modification attacks. The minimum resolution can be set at N=4 to limit the alteration of the AF of watermarked variants. At N=4, a somatic variant AF, which is close to 0, will be moved to 0.125 to switch from Qₒ to Qₓ quantizer. Similarly homozygous AF≈1 will be adjusted to 0.875, while the heterozygous AF≈0.5—to 0.375 or 0.625. The N=4 quantizers introduce a somewhat significant alteration to the AF, but only a small number of variants will be adjusted with the low resolution quantizers. For example, if N is selected randomly between 4 and 50, 87% of modified variants (40 out of 46) will receive quantizers with N>10, or the half-bin size (i.e., maximum displacement) smaller than or equal to 0.05.

### Variant Watermark Insertion Procedure

At each watermarked variant, the resolution N and the quantizer index, I∈{0,1}, is selected deterministically based on a random seed derived from a secret master key. The same sequences of N and I can be reproduced with the same seed to verify the watermark.

If the protected VCF file or the list of variants are merged with other variants, or subsetted, the information about the watermarked variants positions within the N and I sequences, needed to verify the watermark, will be lost. For this reason, the protected variants are securely hashed, and the hash values saved in a small binary file. Specifically, the previously described variant tuple is hashed: genomic position, REF and ALT. To ensure that loci of the shared variants cannot be inferred from the variant hash values, the genomic positions are encrypted prior to hashing (mapped to AES blocks).

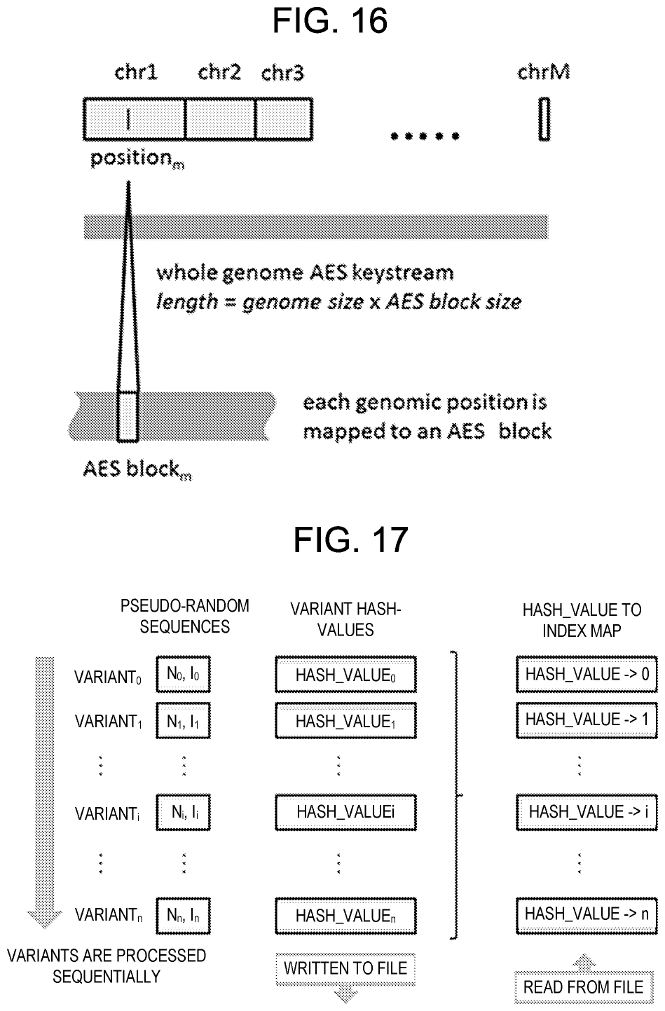

Each genomic position is converted into a single number, an offset from the beginning of the whole genome, ordered by the chromosomal position. The converted genomic positions are then mapped to a whole genome AES keystream, a pseudo-random sequence derived from the master key that covers the whole genome. A unique full AES block (256 bits) corresponds to each genomic position (FIG. 16). Genome Keystream does not need to be fully instantiated, portions of the keystream can be built as needed.

FIG. 16 shows secure mapping of genomic positions to the whole genome AES keystream.

For each watermark variant, REF and ALT strings are concatenated using a separator (e.g., REF=‘A’, ALT=‘ACC’, separator=‘/’: ‘A/ACC’). The concatenated string is combined with the AES block corresponding to the variant position and hashed with the SHA-256 hash function:

hash_value=SHA-256(<REF>‘/’<ALT><AES block>)

The hash values are written into the file sequentially, and the order in which the variants are watermarked is preserved in the file. During watermark discovery, the hash values file will be read, and the variant hash values will be mapped to the order of variants in the pseudo-random sequences N and I (FIG. 17). If an attacker completely removes the AF data (or other data used for watermarking), the saved hash values allows for the determination of whether the protected variants are present in the tested VCF or the variant list, although the quantizer information will be lost. The hash values file contains encrypted loci and can be stored in a public data storage.

FIG. 17 shows example mapping of variants to the quantizer resolution (N) and index (I) pseudo-random sequences.

While all available variants can be used as watermark elements, some variants are skipped to protect against collusion attacks. Similarly to the described BAM watermarking procedure, watermark variants with a high probability p, for example p=0.8, are selected. This ensures that the data recipients get similar but slightly different watermarks. This variant selection is deterministic and the corresponding random seed is derived from the information about the entity receiving the variants. Again, similarly to the described BAM watermarking procedure, it is possible to embed a policy associated with the shared variants into the watermark.

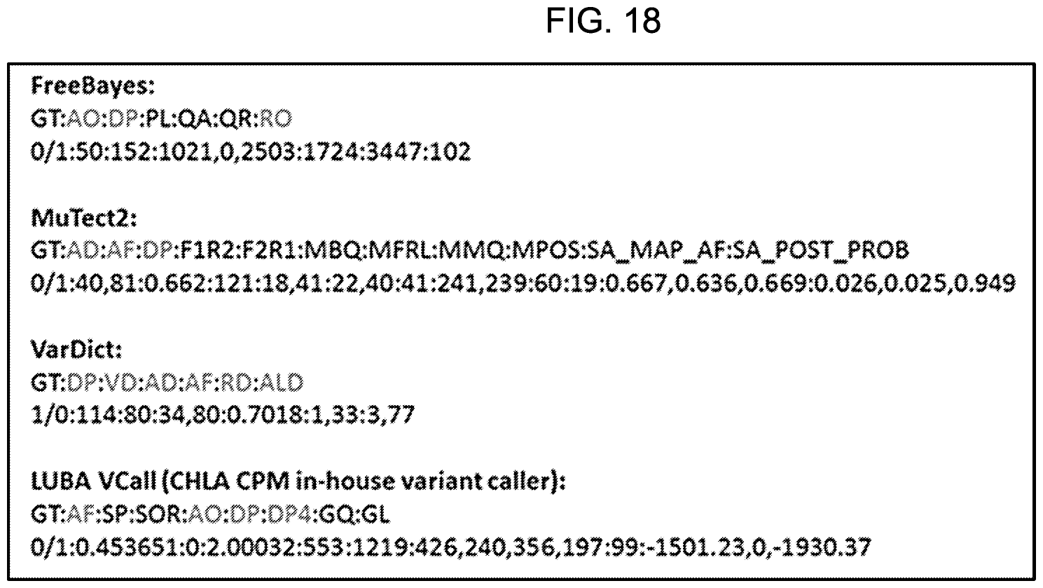

FIG. 18 shows sample genotype data generated by a few selected variant callers. The tags directly related to the AF are highlighted: AO and VD—alternate allele count or variant depth, RO and RD—reference allele count or depth, DP—depth of coverage, ALD and DP4 contain information about forward and reverse strand allele counts.

Data directly related to AF, or more specifically to REF and ALT counts, can be stored using different tags, e.g., AO, DP, RO, AD, RD, etc. (AF-related data is shown in red, see FIG. 18). If AF is modified, all AF-related values must be recalculated, to keep the variant data consistent.

To summarize, the watermark insertion procedure includes the following steps:

Initialization step:
Initialize three pseudo-random number generators with a single seed derived from the master key:
   boolean generator S={true,false}, to select variants for watermarking with high probability, e.g., p=0.8
   integer generator to get quantizer resolution, e.g., N between 4 and 50
   boolean generator to get quantizer index, I={0,1}, p=0.5
      Processing steps for each variant:
Read variant data from the VCF or the variant list, skip variants with low coverage if depth-related data is available (e.g., AO/RO, AD, DP4, etc.)
Get current S, N and I pseudo-random values. Skip variants that are not selected for the watermark.
Hash the selected variant using the master key and write out the hash value.
Check which quantizer index corresponds to the selected variant AF, and adjust AF if it is different from the target quantizer index. Recalculate all AF-related values.
Write out the variant.

### Variant Watermark Discovery Procedure

When a VCF file of a single biological sample is watermarked, the uniqueness of variants defined by the (genomic position, REF, ALT) tuple is guaranteed. For example, multi-allelic variants at the same position will have different REF/ALT combinations. If, however, the watermarked VCF file of a sample is merged with VCF files of other samples, there may be multiple non-unique variants in the combined variant list. These non-unique variants will be ignored during the watermark discovery procedure. Rare variants with low population minor allele frequency (MAF) may be relied upon, to discover a watermark in a large set of merged variants.

To protect against a multiplication attack, when the protected variants are repeated several times, the AF or another value used for watermarking is checked to determine that it is indeed different between the non-unique variants.

To discover the watermark in a given VCF file or a list of variants (which may include additional non-related variants), the following procedure is applied:
Initialization step:
To initialize the discovery: reproduce N and I sequences with the secret key; read the watermarked variants hash values, and create the mapping of the hash values to the variant indices within the N and I sequences.
   Processing steps for each variant:
Check tested variants for uniqueness, drop variants with the same genomic positions and REF/ALT pairs.
For each unique tested variant, calculate the hash value, and search for it in the variant indices map.
If the hash value is not found, the tested variant is skipped. This variant was either present in the original VCF file or the variant list, but not selected for the watermark, or is an unrelated variant added to the protected list.
If the hash value is found in the map, the corresponding variant index m (FIG. 6) is used to get the Nₘ and Iₘ values from the pseudo-random sequences. The quantizers with resolution Nₘ are utilized, the tested variant AF is mapped to one of the quantizers, and the resulting index is checked against Iₘ.
The presence of the watermark will be determined from the counts of matching and mismatching quantizer indices. By chance, if the VCF file or the variant list was not watermarked or watermarked with a different secret key, about the same number of matches and mismatches are expected.

### Extension of Variant Data Watermarking

The method is presented for watermark embedding into the AF data, with the assumption that other parameters that can be used to determine the AF are present as well. In the following sections two special cases are considered. In the first case, a watermark is embedded into an independent variable, e.g., population minor allele frequency (MAF), genotype quality, or AF when other AF-related parameters are not given. In the second case, multiple variables are available for watermarking.

### Special Case 1: An Independent Variable Used for Watermarking

Suppose AF is present without other depth-related data, or a different variable is used for watermarking. In this case, the watermarking variable will be adjusted, when needed, to the nearest end of the target quantizer bin, and a small Gaussian noise will be added, to move it within the target bin:

$${AF}_{x}^{dither} = \{ \begin{matrix}{AF}_{x} + ε , {AF}_{x} > {AF}_{o} \\ {AF}_{x} - ε , {AF}_{x} < {AF}_{o}\end{matrix} , where$$

$$ε = max ( \vert 𝒩 ( 0 , {σ}^{2} ) \vert , \frac{N}{2} ) , {AF}_{o} \to {AF}_{x}$$

The added dithering noise will obscure the specific quantizers resolution, so that an attacker cannot guess what it is, and use this information to remove watermark elements.

An independent variable may be defined within a range different from [0,1], e.g., only somatic variants with 0<AF<0.2 may be considered. The same reasoning as presented for the [0,1] range can be applied for other intervals without the loss of generality.

### Special Case 2: Multiple Variables are Available for Watermarking

Suppose a VCF file or a variant database that contains population minor allele frequency or frequencies is shared. For example, a gnomAD-like (Genome Aggregation Database) VCF file, that incorporates allele frequencies for different populations and genders:
##INFO=<ID=AF,Number=A,Type=Float,Description=“Allele Frequency among genotypes, for each ALT allele, in the same order as listed”>
##INFO=<ID=AF_AFR,Number=A,Type=Float,Description=“ Allele Frequency among African/African American genotypes, for each ALT allele, in the same order as listed”>
##INFO=<ID=AF_AMR,Number=A,Type=Float,Description=“Allele Frequency among Admixed American genotypes, for each ALT allele, in the same order as listed”>
##INFO=<ID=AF_ASJ,Number=A,Type=Float,Description=“Allele Frequency among Ashkenazi Jewish genotypes, for each ALT allele, in the same order as listed”>
##INFO=<ID=AF_EAS,Number=A,Type=Float,Description=“Allele Frequency among East Asian genotypes, for each ALT allele, in the same order as listed”>
##INFO=<ID=AF_FIN,Number=A,Type=Float,Description=“Allele Frequency among Finnish genotypes, for each ALT allele, in the same order as listed”>
##INFO=<ID=AF_NFE,Number=A,Type=Float,Description=“Allele Frequency among Non-Finnish European genotypes, for each ALT allele, in the same order as listed”>
##INFO=<ID=AF_OTH,Number=A,Type=Float,Description=“Allele Frequency among Other (population not assigned) genotypes, for each ALT allele, in the same order as listed”>
##INFO=<ID=AF_Male,Number=A,Type=Float,Description=“Allele Frequency among Male genotypes, for each ALT allele, in the same order as listed”>
##INFO=<ID=AF_Female,Number=A,Type=Float,Description=“Allele Frequency among Female genotypes, for each ALT allele, in the same order as listed”>
##INFO=<ID=AF_POPMAX,Number=A,Type=Float,Description=“Maximum Allele Frequency across populations (excluding OTH)”>

In this example, most of these variables can be quantized separately, and multiple bits can be embedded in each variant. In general, if some variables are not independent (e.g., AF and AF_POPMAX in this example), or are under constrains (e.g., need to add up to 1), the number of variables available for watermarking will be reduced. Not all variables may be available for all variants, and in one example, up to 9 bits may be able to be embedded in each variant. A probability of discovering all nine target quantizers in a variant by chance, i.e., p(I=0)=p(I=1)=0.5, is very low: 0.5⁹=0.002. Therefore, when multiple variables are available for watermarking, in some cases a small number of variants or even a single variant can carry a discoverable watermark.

### Example Embodiments

A first example includes a method of dynamically applying a watermark to at least a portion of a file, the method comprising generating, using information derived from a secret key, a first random seed; generating, using the first random seed, an ordered pseudorandom set of integers; generating, using dynamic attribute information, a second random seed; selecting, using the second random seed, a subset of the ordered pseudorandom set of integers, the subset corresponding to identifiers of data locations in the file; and modifying data at data locations in the file corresponding to at least a portion of the identifiers included in the subset to generate a watermarked file.

A second example includes the first example, and further includes the method, wherein the dynamic attribute information includes entity information for an entity to which the file is being distributed to or shared with, timing information corresponding to a validity time period for accessing the file, a data usage policy for the file, and/or one or more other attributes of a policy for the data.

A third example includes the first and/or second examples, and further includes the method, wherein the modifying the data comprises generating, using the first random seed, a pseudorandom integer and changing the data to a value that is based on the pseudorandom integer.

A fourth example includes one or more of the first through third examples, and further includes determining which of the data locations corresponding to the identifiers of the subset meet selected criteria, and wherein the portion of the identifiers correspond to the identifiers of the subset that meet the selected criteria.

A fifth example includes one or more of the first through fourth examples, and further includes assigning a selected quality score to modified data, the selected quality score being selected based on quality scores of data at each other data location in the file.

A sixth example includes one or more of the first through fifth examples, and further includes the method, wherein the selected quality score corresponds to a quality score below a threshold that is most frequently assigned to the data at each other data location in the file relative to other quality scores below the threshold.

A seventh example includes one or more of the first through sixth examples, and further includes the method, wherein the first random seed and/or the second random seed is generated with a secure hash algorithm.

An eighth example includes one or more of the first through seventh examples, and further includes the method, wherein an entity to which the file is being distributed is a first entity, and wherein the subset of the ordered pseudorandom set of integers is selected to only partially overlap with another subset or subsets of ordered pseudorandom sets of integers that is generated for watermarking the file for distribution to another, different entity or entities.

A ninth example includes one or more of the first through eighth examples, and further includes the method, wherein the file comprises a genomic data file that includes a sequencing data set.

A tenth example includes one or more of the first through ninth examples, and further includes the method, wherein the genomic data file is a Binary Alignment Map (BAM) file.

An eleventh example includes one or more of the first through tenth examples, and further includes the method, wherein the data locations in the file comprise reference bases in the sequencing data set, and wherein modifying the data comprises switching the reference bases at the data locations in the file corresponding to at least the portion of the identifiers included in the subset from the respective reference base to a selected alternative base.

A twelfth example includes one or more of the first through eleventh examples, and further includes the method, wherein the selected alternative base is selected based on a randomly generated number that is generated using the first random seed.

A thirteenth example includes one or more of the first through twelfth examples, and further includes determining which of the data locations corresponding to the identifiers of the subset meet selected criteria, wherein the portion of the identifiers correspond to the identifiers of the subset that meet the selected criteria, and wherein the selected criteria includes data locations that have a number of sequencing reads with the selected alternative base that is less than a threshold.

A fourteenth example includes one or more of the first through thirteenth examples, and further includes the method, wherein the watermarked file is a reference watermarked file, the method further comprising validating a targeted file by determining whether the watermark is present in the targeted file by generating a sequence of watermark elements based on information derived from the secret key and comparing the percentage of watermark elements discovered in the targeted file to the expected percentage of watermark elements that can be discovered by chance, estimated by a Monte Carlo simulation with random seeds.

A fifteenth example includes one or more of the first through fourteenth examples, and further includes detecting collusion between two or more entities to attempt to modify or remove a watermark from the file by determining which watermark elements in the sequence of watermark elements generated during generation of the reference watermarked file are missing in the targeted file and which watermark elements in the sequence of watermark elements generated during generation of the reference watermarked file are present in the targeted file.

A sixteenth example includes one or more of the first through fifteenth examples, and further includes transmitting the watermarked file to an entity that satisfies the dynamic attribute information.

A seventeenth example includes one or more of the first through sixteenth examples, and further includes dynamically encrypting the watermarked file.

An eighteenth example includes one or more of the first through seventeenth examples, and further includes the method, wherein the secret key is a watermarking secret key and the watermarked filed is formed of multiple blocks of ordered data to enable partial decryption of the watermarked file, and wherein dynamically encrypting the watermarked file comprises generating, using an encryption secret key and one or more initialization vectors associated with the watermarked file, a keystream for the multiple blocks of ordered data of the watermarked file; encrypting the multiple blocks of ordered data of the watermarked file by performing a logical operation of the keystream with the multiple blocks of ordered data in a one-to-one correspondence; and building a file index of the watermarked file to identify location information of the multiple blocks of ordered data.

A nineteenth example includes one or more of the first through eighteenth examples, and further includes the method, wherein the keystream is formed of a plurality of blocks, each block of the keystream corresponding to an associated block of the watermarked file.

A twentieth example includes one or more of the first through nineteenth examples, and further includes the method, wherein each block of the keystream has a value that is a function of the encryption secret key, the initialization vectors, and an offset of the respective associated block of the file from a beginning of the file, and wherein each block of the keystream has a length that is equal to a length of the respective associated block of the file, wherein the initialization vectors include a value that is combined with the encryption secret key to generate the keystream.

A twenty-first example includes one or more of the first through twentieth examples, and further includes the method, wherein building the index of the file comprises, for each block of the watermarked file: reading the block from the watermarked file, wherein the ordered data of the block includes one or more data groupings; identifying start and end positions for each data grouping of the block and saving the start and end positions with an associated read offset from a start of the block; updating a block encryption index for the block, the block encryption index identifying the start and end positions of the data groupings for the block; and updating the file index for the watermarked file using the saved start and end positions and the associated read offsets identified in the block encryption index, the file index storing the information from the block encryption index for each block of the watermarked file.

A twenty-second example includes one or more of the first through twenty-first examples, and further includes the method, wherein the data groupings include sorted genomic sequencing data.

A twenty-third example includes one or more of the first through twenty-second examples, and further includes the method, wherein the sorted genomic sequencing data is sorted by chromosome position.

A twenty-fourth example includes one or more of the first through twenty-third examples, and further includes the method, wherein each of the associated read offsets comprises a respective number of bits or a respective number of bytes indicating a distance from a beginning of the file.

A twenty-fifth example includes one or more of the first through twenty-fourth examples, and further includes the method, wherein the encryption secret key and/or the keystream is generated using a stream cipher or a block cipher in a counter mode of operation.

A twenty-sixth example includes one or more of the first through twenty-fifth examples, and further includes the method, wherein the stream cipher includes Salsa 20 and wherein the block cipher in the counter mode of operation includes Advanced Encryption Standard, Counter mode (AES-CTR).

A twenty-seventh example includes one or more of the first through twenty-sixth examples, and further includes the method, wherein the watermarked file is an ordered genomic sequencing data file.

A twenty-eighth example includes one or more of the first through twenty-seventh examples, and further includes the method, wherein the ordered genomic data file is in a Blocked GNU Zip Format (BGZF).

A twenty-ninth example includes one or more of the first through twenty-eighth examples, and further includes the method, wherein the ordered genomic data file is a Binary Alignment Map (BAM) file storing genomic sequences or a Variant Call Format (VCF) file storing genomic variation.

A thirtieth example includes one or more of the first through twenty-ninth examples, and further includes the method, wherein the logical operation includes an XOR or an XNOR operation.

A thirty-first example includes one or more of the first through thirtieth examples, and further includes the method, wherein the encryption secret key is a random number that is not shared during decryption of the file.

A thirty-second example includes one or more of the first through thirty-first examples, and further includes the method, wherein dynamically encrypting the watermarked file includes encrypting only a portion of the watermarked file, encrypting different portions of the watermarked file at different times, encrypting only a portion of a block of the watermarked file, and/or re-encrypting at least a portion of the watermarked file after performing a prior encryption of the watermarked file.

A thirty-third example includes one or more of the first through thirty-second examples, and further includes embedding policy information in the encrypted blocks of data, the policy information defining, for each data grouping of each block of the watermarked file, rules for decrypting the data grouping.

A thirty-fourth example includes one or more of the first through thirty-third examples, and further includes the method, wherein the rules include time-based rules that define a time or time duration in which the data grouping is allowed to be decrypted, requesting party rules that define entities and/or users that are allowed to the data, and/or usage rules that define one or more usages for which the data is allowed to be decrypted or accessed.

A thirty-fifth example includes one or more of the first through thirty-fourth examples, and further includes revising one or more of the rules for decrypting the data grouping responsive to receiving an associated request from an owner of the ordered data stored in the watermarked file.

A thirty-sixth example includes one or more of the first through thirty-fifth examples, and further includes the method, wherein revising one or more of the rules includes rescinding access to one or more portions of the keystream and/or rescinding, after at least a portion of the watermarked file is decrypted, access to decrypted data of the watermarked file.

A thirty-seventh example includes one or more of the first through thirty-sixth examples, and further includes the method, wherein encrypting the multiple blocks of ordered data generates multiple blocks of encrypted data corresponding to the watermarked file, the method further comprising dynamically decrypting at least a portion of the watermarked file.

A thirty-eighth example includes one or more of the first through thirty-seventh examples, and further includes the method wherein dynamically decrypting at least the portion of the watermarked file includes decrypting at least one selected block of encrypted data of the watermarked file using a portion of the keystream, the portion of the keystream corresponding to the at least one selected block.

A thirty-ninth example includes one or more of the first through thirty-eighth examples, and further includes the method, wherein the at least one selected block of encrypted data comprises only a subset of the multiple blocks of encrypted data of the watermarked file.

A fortieth example includes one or more of the first through thirty-ninth examples, and further includes the method, wherein decrypting the at least one selected block includes performing a logical operation of the portion of the keystream with the encrypted data of the at least one selected block to generate plaintext data corresponding only to the at least one selected block.

A forty-first example includes one or more of the first through fortieth examples, and further includes the method, wherein the genomic data file is a Variant Call Format (VCF) file or a list of variants storing genomic variation data, and wherein the watermarks are embedded in variant allele frequency and/or other rational data associated with the variants.

A forty-second example includes one or more of the first through forty-first examples, and further includes the method, wherein the variant allele frequency is included in the genomic variation data and/or wherein the variant allele frequency is calculated based on an alternative alleles count for the genomic variation data and a depth of coverage at a variant position or a count of reference alleles for the genomic variation data.

A forty-third example includes one or more of the first through forty-second examples, and further includes dividing a range of the variant allele frequency into a plurality of bins of size 1/N and shifting the bins by a half-length of 1/(2N), where a first bin and a last bin are each of size 1/(2N); assigning adjacent bins to a respective different one of two quantizers; selecting, for each variant position in the genomic variation data, a target bin size and a target quantizer index based on the secret key; and for each variant in the genomic variation data having a depth of coverage above a threshold, adjusting an alternative allele count such that a corresponding allele frequency for the variant falls into a selected one of the plurality of bins corresponding to the selected target bin size and target quantizer index.

A forty-fourth example includes one or more of the first through forty-third examples, and further includes the method, wherein N is set to an integer greater than one, to preserve variant genotypes.

A forty-fifth example includes one or more of the first through forty-second examples, and further includes randomly selecting N from a range of numbers, wherein minimum and maximum values of the range correspond to lowest and highest resolution of quantizers, respectively.

A forty-sixth example includes one or more of the first through forty-fifth examples, and further includes securely hashing variant tuples of the genomic variation data to generate a plurality of hash values.

A forty-seventh example includes one or more of the first through forty-sixth examples, and further includes storing the hash values in a binary file.

A forty-eighth example includes one or more of the first through forty-seventh examples, and further includes encrypting genomic positions of the variant tuples prior to securely hashing the variant tuples.

A forty-ninth example includes a method of detecting and/or verifying a watermark in a file, the method comprising generating, using information derived from a secret key associated with the watermark, a first random seed; generating, using the first random seed, an ordered pseudorandom set of integers; generating, using entity information for at least one entity to which the file was distributed and timing information corresponding to a validity time period for the file, a second random seed; selecting, using the second random seed, a subset of the ordered pseudorandom set of integers, the subset corresponding to identifiers of genomic data locations; generating a sequence of watermark elements, the watermark elements comprising expected values for associated locations in the file, the associated locations being selected based on the first random seed and the expected values being selected based on the second random seed; and comparing the sequence of watermark elements to the file to determine whether the associated locations in the file are populated with the respective associated expected values.

A fiftieth example includes the forty-ninth example and further includes the method, wherein the file is an encrypted file formed of multiple blocks of encrypted data, the method further comprising dynamically decrypting at least a portion of the file to generate a decrypted file, and wherein comparing the sequence of watermark elements to the file comprises comparing the sequence of watermark elements to the decrypted file.

A fifty-first example includes the forty-ninth and/or fiftieth examples, and further includes the method, wherein dynamically decrypting at least a portion of the file comprises: receiving a request to decrypt at least one selected block of encrypted data of the file; responsive to validating the request, retrieving a portion of a keystream for the file, the portion of the keystream corresponding to the at least one selected block; and decrypting the at least one selected block by performing a logical operation of the portion of the keystream with the encrypted data of the at least one selected block to generate plaintext data corresponding only to the at least one selected block.

A fifty-second example includes one or more of the forty-ninth through fifty-first examples, and further includes validating the request by comparing attributes of the request and a user making the request with one or more attributes associated with the user and/or policies bound with the encrypted data to determine if the user and the request are in compliance with the attributes and policies, respectively.

A fifty-third example includes one or more of the forty-ninth through fifty-second examples, and further includes the method, wherein dynamically decrypting the file comprises decrypting selected portions of the file using the keystream while remaining portions of the file are not decryptable.

A fifty-fourth example includes one or more of the forty-ninth through fifty-third examples, and further includes the method, wherein selected portions of the file are decryptable using the portion of the keystream while remaining portions of the file are not decryptable.

A fifty-fifth example includes one or more of the forty-ninth through fifty-fourth examples, and further includes the method, wherein the encrypted data of the file is generated using an encryption secret key, the encryption secret key being used to generate the keystream, different portions of which are subsequently used for decrypting only respective portions of the file in respective decryption iterations without sharing the encryption secret key.

A fifty-sixth example includes one or more of the forty-ninth through fifty-fifth examples, and further includes the method, wherein the file is a Variant Call Format (VCF) file storing genomic variation data.

A fifty-seventh example includes method of inserting a watermark into a Variant Call Format (VCF) file or into a list of variants, the method comprising initializing three pseudo-random number generators with a single seed derived from a master key; reading variant data from the VCF or a variant list; determining a pseudo-random value for each of the three pseudo-random number generators; selecting variants from the variant data for watermarking based on a first generator of the three pseudo-random number generator; for each selected variant: hashing the selected variant using the master key and writing out the hash value; determining a quantizer index that corresponds to an allele frequency of the selected variant and adjusting the allele frequency to fit a quantizer bin associated with the quantizer index; recalculating values relating to allele frequency; and writing out the variant based on the recalculated values.

A fifty-eighth example includes the fifty-seventh example, and further includes the method, wherein the pseudo-random number generators include a first, Boolean generator for selecting variants for watermarking; a second, integer generator for selecting quantizer resolutions, and a third, Boolean generator for selecting quantizer indices.

A fifty-ninth example includes the fifty-seventh example and/or the fifty-eighth example, and further includes the method, wherein reading the variant data comprises only reading variant data for variants with depth above a threshold.

A sixtieth example includes a method of detecting and/or verifying a watermark in a Variant Call Format (VCF) file or a list of variants, the method comprising generating, using information derived from a secret key associated with the watermark, a first sequence of pseudo-random numbers and a second sequence of pseudo-random numbers; reading hash values for watermarked variants of the VCF file; creating a mapping of the hash values to variant indices within the first and second sequences of pseudo-random numbers to generate a variant indices map; checking tested variants for uniqueness and dropping variants with the same genomic positions and reference/alternate alleles pairs; for each unique tested variant, calculating a corresponding tested hash value and searching for the calculated tested hash value in the variant indices map; for each calculated tested hash value found in the variant indices map, using a corresponding variant index m to determine Nm and Im values from the first and second sequences of pseudo-random numbers respectively, using quantizers with resolution Nm, mapping a tested variant allele frequency corresponding to the variant index m to one of the quantizers to determine a resulting index, and comparing the resulting index to Im; and determining a presence of the watermark based on counts of matching and mismatching quantizer indices.

A sixty-first example includes a method of inserting a watermark into a FASTQ file, the method comprising aligning sequence reads in the FASTQ file to a reference sequence to create a BAM file; inserting the watermark with the method in example 10; and converting the BAM file back to the FASTQ file.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of dynamically applying a watermark to at least a portion of a file, the method comprising:
generating, using information derived from a secret key, a first random seed;
generating, using the first random seed, an ordered pseudorandom set of integers;
generating, using dynamic attribute information, a second random seed;
selecting, using the second random seed, a subset of the ordered pseudorandom set of integers, the subset corresponding to identifiers of data locations in the file; and
modifying data at data locations in the file corresponding to at least a portion of the identifiers included in the subset to generate a watermarked file.

2. The method of claim 1, wherein the dynamic attribute information includes entity information for an entity to which the file is being distributed to or shared with, timing information corresponding to a validity time period for accessing the file, a data usage policy for the file, and/or one or more other attributes of a policy for the data.

3. The method of claim 1, wherein the genomic data file is a Variant Call Format (VCF) file or a list of variants storing genomic variation data, and wherein the watermarks are embedded in variant allele frequency and/or other rational data associated with the variants.

4. The method of claim 3, wherein the variant allele frequency is included in the genomic variation data and/or wherein the variant allele frequency is calculated based on an alternative alleles count for the genomic variation data and a depth of coverage at a variant position or a count of reference alleles for the genomic variation data.

5. The method of claim 3, further comprising:
dividing a range of the variant allele frequency into a plurality of bins of size 1/N and shifting the bins by a half-length of 1/(2N), where a first bin and a last bin are each of size 1/(2N);
assigning adjacent bins to a respective different one of two quantizers;
selecting, for each variant position in the genomic variation data, a target bin size and a target quantizer index based on the secret key; and
for each variant in the genomic variation data having a depth of coverage above a threshold, adjusting an alternative allele count such that a corresponding allele frequency for the variant falls into a selected one of the plurality of bins corresponding to the selected target bin size and target quantizer index.

6. The method of claim 5, wherein N is set to an integer greater than one, to preserve variant genotypes.

7. The method of claim 5, further comprising randomly selecting N from a range of numbers, wherein minimum and maximum values of the range correspond to lowest and highest resolution of quantizers, respectively.

8. The method of claim 3, further comprising securely hashing variant tuples of the genomic variation data to generate a plurality of hash values.

9. The method of claim 8, further comprising storing the hash values in a binary file.

10. The method of claim 8, further comprising encrypting genomic positions of the variant tuples prior to securely hashing the variant tuples.

11. A method of inserting a watermark into a Variant Call Format (VCF) file or into a list of variants, the method comprising:
initializing three pseudo-random number generators with a single seed derived from a master key;
reading variant data from the VCF or a variant list; determining a pseudo-random value for each of the three pseudo-random number generators;
selecting variants from the variant data for watermarking based on a first generator of the three pseudo-random number generator;
for each selected variant:
hashing the selected variant using the master key and writing out the hash value;
determining a quantizer index that corresponds to an allele frequency of the selected variant and adjusting the allele frequency to fit a quantizer bin associated with the quantizer index;
recalculating values relating to allele frequency; and
writing out the variant based on the recalculated values.

12.The method of claim 11, wherein the pseudo-random number generators include a first, Boolean generator for selecting variants for watermarking; a second, integer generator for selecting quantizer resolutions, and a third, Boolean generator for selecting quantizer indices.

13. The method of claim 11, wherein reading the variant data comprises only reading variant data for variants with depth above a threshold.

14. A method of detecting and/or verifying a watermark in a Variant Call Format (VCF) file or a list of variants, the method comprising:
generating, using information derived from a secret key associated with the watermark, a first sequence of pseudo-random numbers and a second sequence of pseudo- random numbers;
reading hash values for watermarked variants of the VCF file;
creating a mapping of the hash values to variant indices within the first and second sequences of pseudo-random numbers to generate a variant indices map;
checking tested variants for uniqueness and dropping variants with the same genomic positions and reference/alternate alleles pairs;
for each unique tested variant, calculating a corresponding tested hash value and searching for the calculated tested hash value in the variant indices map;
for each calculated tested hash value found in the variant indices map, using a corresponding variant index m to determine Nm and Im values from the first and second sequences of pseudo-random numbers respectively, using quantizers with resolution Nₘ, mapping a tested variant allele frequency corresponding to the variant index m to one of the quantizers to determine a resulting index, and comparing the resulting index to Im; and
determining a presence of the watermark based on counts of matching and mismatching quantizer indices.

15. The method of claim 14, wherein the file is an encrypted file formed of multiple blocks of encrypted data, the method further comprising dynamically decrypting at least a portion of the file to generate a decrypted file, and wherein comparing the sequence of watermark elements to the file comprises comparing the sequence of watermark elements to the decrypted file.

16. The method of claim 15, wherein dynamically decrypting at least a portion of the file comprises:
receiving a request to decrypt at least one selected block of encrypted data of the file;
responsive to validating the request, retrieving a portion of a keystream for the file, the portion of the keystream corresponding to the at least one selected block; and
decrypting the at least one selected block by performing a logical operation of the portion of the keystream with the encrypted data of the at least one selected block to generate plaintext data corresponding only to the at least one selected block.

17. The method of claim 16, further comprising validating the request by comparing attributes of the request and a user making the request with one or more attributes associated with the user and/or policies bound with the encrypted data to determine if the user and the request are in compliance with the attributes and policies, respectively.

18. The method of claim 16, wherein dynamically decrypting the file comprises decrypting selected portions of the file using the keystream while remaining portions of the file are not decryptable.

19. The method of claim 16, wherein selected portions of the file are decryptable using the portion of the keystream while remaining portions of the file are not decryptable.

20. The method of claim 14, wherein the encrypted data of the file is generated using an encryption secret key, the encryption secret key being used to generate the keystream, different portions of which are subsequently used for decrypting only respective portions of the file in respective decryption iterations without sharing the encryption secret key.
